(19) United States
(12) Patent Application Publication
(10) Pub. No.: US 2010/0174213 A1
(43) Pub. Date: Jul. 8, 2010

(54) Audio relaxing systems and methods

(76) Inventor: Youngtack Shim, Port Moody (CA)

Correspondence Address: Youngtach Shim, 155 Aspenwood Drive, Port Moody, BC, V3H 5A5, CA

(21) Appl. No.: 12/318,663

(22) Filed: Jan. 6, 2009

(51) Int. Cl. A61B 5/00; A61B 5/103; A61B 19/00

(52) U.S. Cl. 600/587; 600/38; 128/898

(57) ABSTRACT

The present invention generally relates to pelvic relaxing systems capable of generating sound of an user or third party or capable of being manipulated by the sound. More particularly, the present invention relates to pelvic relaxing systems for acquiring real-time or pre-recorded sounds, playing the sounds during use or in response to an user input, synchronizing the sounds with movements and/or operations of the system, and playing the sound synthesized from multiple audio signals with different content and/or voice bases. Such systems may be controlled so that the movements of various parts and/or operations thereof may be controlled by or synchronized with the sounds provided thereto by audio and communication devices. The present invention also relates to various methods of obtaining such sounds, making and using such sounds, synchronizing such sounds with the movements of the parts, and controlling such movements according to such sounds. The present invention also relates to various processes for making various members, units, and parts of the pelvic relaxing systems, for synchronizing such with the sounds, and for making such controlled by such sounds.

### CROSS-REFERENCES TO RELATED APPLICATIONS

The present application claims an earlier invention date of the Disclosure Document entitled the same, deposited in the U.S. Patent and Trademark Office (the “Office”) on Jan. 23, 2007 under the Disclosure Document Deposit Program (“DDDP”) of the Office, and bearing the Ser. No. 611,331. The present application also claims an earlier invention date of another Disclosure Documents which is entitled “Dynamic control relaxing systems and methods,” deposited in the Office on Jan. 12, 2007 under the DDDP, and bearing the Ser. No. 611,023. It is to be appreciated that an entire portion of each of the above Disclosure Documents is incorporated herein by reference.

### FIELD OF THE INVENTION

The present invention generally relates to pelvic relaxing systems capable of generating sound of an user or third party or capable of being manipulated by the sound. More particularly, the present invention relates to pelvic relaxing systems for acquiring real-time or pre-recorded sounds, playing the sounds during use or in response to an user input, synchronizing the sounds with movements and/or operations of the system, and playing the sound synthesized from multiple audio signals with different content and/or voice bases. Such systems may be controlled so that the movements of various parts and/or operations thereof may be controlled by or synchronized with the sounds provided thereto by audio and communication devices. The present invention also relates to various methods of obtaining such sounds, making and using such sounds, synchronizing such sounds with the movements of the parts, and controlling such movements according to such sounds. The present-invention also relates to various processes for making various members, units, and parts of the pelvic relaxing systems, for synchronizing such with the sounds, and for making such controlled by such sounds.

### BACKGROUND OF THE INVENTION

A vaginal anatomy typically includes a vaginal entry and a vaginal wall, where such an entry defines an orifice therethrough, while the wall includes muscles and defines a vaginal cavity which extends inwardly from the entry and which is also bound by such muscles. The entry forms a clitoris thereon, and a paraurethral gland of an urethral sponge of a clitoris (also called the Grafenberg spot or G spot) is believed to be defined on the wall. The vaginal wall is formed essentially of two sets of muscles, the former extending longitudinally while the latter encircling the vagina. These muscles are specifically termed as “pubococygenus” and “levator ani” and are located immediately adjacent to the vagina. These muscles have general appearance of a hammock with its two ends connected to the sides of a pelvis. In particular, the pubococygenus is basically a sphincter muscle which passes through a middle third of the vagina and runs in a circular band, with a ring-like ridges forming a part of a urethra and anus. For simplicity of illustration, this vaginal anatomy is to be referred to as “a pelvic structure,” the vaginal entry as an “entry,” and the vaginal wall as a “wall.” In addition, such a pelvic structure is referred to as the “standard pelvic structure” or simply the “pelvic structure” hereinafter for simplicity of illustration, unless otherwise specified.

Among devices currently available in the market for enhancing sexual functioning are dildos, vaginal exercise bars, and prostate stimulators. These devices generally provide stimuli from friction upon manipulation of their stimulators or by pressure due to distension of the pelvic cavity effected by a volume of such stimulators.

For example, manual pelvic relaxing devices have been proposed in various configurations as disclosed in various prior art such as, e.g., U.S. Pat. No. 3,996,930 to Sekulich, U.S. Pat. No. 5,690,603 to Kain, U.S. Pat. No. 5,690,604 to Barnett, U.S. Pat. No. 5,853,362 to Jacobs, U.S. Pat. No. 6,203,491 to Uribe, U.S. Pat. App. Pub. No. 2005/0187431 by Hudson, U.S. Pat. App. Pub. No. 2005/0228218 by Skidmore et al., U.S. Pat. No. 6,540,667 to Hickman, and the like. Being manual, users have to manually move such devices in and out of the pelvic cavity and/or around the pelvic opening.

In order to overcome inconvenience thereof, various automatic mechanisms have been added to the pelvic relaxation devices. In one class of examples, electric motors or electromagnetic vibration mechanisms have been incorporated to various automatic devices for effecting vibration as disclosed in various prior art such as, e.g., U.S. Pat. No. 3,451,391 to Tavel, U.S. Pat. No. 3,504,665 to Bakunin et al., U.S. Pat. No. 3,626,931 to Bysakh, U.S. Pat. No. 3,669,100 to Csanad, U.S. Pat. No. 3,991,751 to O'Rourke, U.S. Pat. No. 4,788,968 to Rudashevsky et al., U.S. Pat. No. 5,067,480 to Woog et al, U.S. Pat. No. 6,056,705 to Stigar-Brown, U.S. Pat. Appl. Pub. 2004/0034315 to Chen, U.S. Pat. Appl. Pub. 2004/0127766 to Chen, and the like.

In another class of example, automatic pelvic relaxing devices have used various mechanisms of converting rotational movements generated by such electric motors into translational movements for effecting horizontal and/or vertical translation of their stimulators. Several examples of such prior art include U.S. Pat. No. 4,722,327 to Harvey, U.S. Pat. No. 4,790,296 to Segal, U.S. Pat. No. 5,076,261 to Black, U.S. Pat. No. 5,725,473 to Taylor, U.S. Pat. No. 6,142,929 to Padgett, U.S. Pat. No. 6,422,993 to Hudson, U.S. Pat. No. 6,866,645 to Lee, and U.S. Pat. Appl. Pub. 2004/0147858 Various automatic pelvic relaxing devices have also used various mechanisms for converting rotational movements generated by such electric motors into lateral movements for effecting horizontal translation of their stimulators along a direction generally normal to axes of such stimulators. Several examples of such prior art are U.S. Pat. No. 5,460,597 to Hopper, U.S. Pat. No. 5,470,303 to Leonard et al., and U.S. Pat. No. 5,851,175 to Nickell.

Other therapeutic devices, although developed for various purposes other than pelvic relaxing, seem to have been used as alternatives as such conventional pelvic relaxing devices. In one class of examples, various manual or vibration devices have been disclosed to train or heal pelvic muscles as exemplified in U.S. Pat. No. 3,598,106 to Buning, U.S. Pat. No. 4,241,912 to Mercer et al., and U.S. Pat. No. 4,574,791 to Mitchener. In another class of examples, various devices have been developed for massaging various portions of a human body as disclosed in U.S. Pat. No. 4,055,170 to Nohmura, U.S. Pat. No. 4,825,853 to Iwamoto et al., U.S. Pat. No. 4,846,158 to Teranishi, U.S. Pat. No. 4,911,149 to Borodulin et al., U.S. Pat. No. 5,063,911 to Teranishi, and the like. In another class, massage devices have also been devised to provide translational movements as disclosed in U.S. Pat. No. 4,002,164 to Bradley, U.S. Pat. No. 5,085,207 to Fiore, U.S. Pat. No. 5,676,637 to Lee, and the like. In another class of examples, various devices have also been arranged to provide rotating, tapping, swinging and/or swivelling movements as described in U.S. Pat. No. 4,162,675 to Kawada, U.S. Pat. No. 6,632,185 to Chen, U.S. Pat. No. 4,088,128 to Mabuchi, U.S. Pat. No. 4,513,737 to Mabuchi, U.S. Pat. No. 4,827,914 to Kamazawa, U.S. Pat. No. 4,834,075 to Guo et al., U.S. Pat. No. 5,183,034 to Yamasaki et al., and U.S. Pat. No. 6,402,710 to Hsu. A vacuum device of U.S. Pat. No. 4,033,338 to lgwebike as well as a balloon device of U.S. Pat. No. 4,050,449 to Castellana et al. have also been proposed.

Regardless of their detailed mechanisms and/or movements effected thereby, all of these prior art devices suffer from common drawbacks. Excluding those manual ones, typical automatic devices consist of main modules and control modules which operatively couple with the main modules by wire for delivering electric power and control signals. Such wire, however, tends to be easily tangled and damaged. To overcome this defect, modern automatic pelvic relaxing device are fabricated as single unitary articles each with a main body and a handle which fixedly couples with a top part of the main body. The main body is generally designed to be inserted into the pelvic cavity, whereas the handle is shaped and sized to provide a grip for the user and also incorporates therein various control buttons. Accordingly, the handle consists of a space to form the grip and another space for such buttons. In order to avoid providing an inadvertently long device, however, a part of the handle closer to the main body is recruited to define the grip, whereas the rest of the handle houses the control buttons. It is to be appreciated, however, that all control buttons of conventional automatic devices are either on/off switches or speed control switches, where the on/off switches turn on and off the entire device or a specific movement thereof, and the speed control switches control a speed of the specific movement. In addition, such switches are typically designed to be activated and deactivated each time the user presses or touches them. Accordingly, when the user inadvertently touches any of such on/off and control switches during use, the device may be accidentally turned off, change speeds, and the like. In order to avoid such inadvertent operation, the control buttons have been incorporated as far away from the grip space of the handle, which in turn causes the very inconvenience of requiring the user to change the grip or to move his or her hand to manipulate the control buttons during operation when the user wants to change the speed of movement.

In contrary to these devices, novel pelvic relaxing systems, methods, and/or processes have already been conceived of and disclosed in numerous co-pending Applications of the same Applicant. For example, various pelvic relaxing systems have been proposed for manipulating their input and/or sensor units without mandating the user to change the grip, for providing various stimuli to the clitoris and/or G-spot of the user, for providing interactive capabilities thereto, for synchronizing movements of their various parts and/or operations thereof with internal and/or external signals, for incorporating electric stimulators thereinto, for installing the body members capable of adjusting their configurations, for incorporating retention mechanisms thereinto, for providing feedback mechanisms thereto, and for generating reciprocating movements of only portions of body members thereof. Although these novel systems solve most deficiencies of the conventional devices, none of them are capable of providing audible effects during their use.

Therefore, there is a need for a pelvic relaxing system capable of generating sound of an user or third party during operation of the system and/or capable of manipulating its operation by the sound.

### SUMMARY OF THE INVENTION

The present invention generally relates to pelvic relaxing systems capable of generating sound of an user or third party or capable of being manipulated by the sound. More particularly, the present invention relates to pelvic relaxing systems for acquiring real-time and/or pre-recorded sounds from a third party and/or an user and playing the sounds during use and/or in response to an user input, for synchronizing such sounds with movements of various parts of the systems and/or operations of the systems, for playing the sounds synthesized from multiple audio signals each defining different voice bases and/or content bases, and so on. The present invention also relates to pelvic relaxing systems of which operations may be controlled at least in part by such sounds so that movements of various parts and/or operations thereof may be controlled by and/or synchronized with the sounds which are provided thereto by audio and/or communication devices.

The present invention also relates to various methods of obtaining such sounds from various sources internal or external to such systems, playing such sounds in response to various operations of the systems, playing one or more of such sounds depending upon types of the movements of the parts of the system, synchronizing such sounds with various movements of such parts, types of the movements of such parts, and/or parts of the system recruited for such movements, selecting one or more parts of the system for such movements designated by the user input, and so on. The present invention also relates to various methods of manipulating such movements based upon such sounds, selecting one or multiple parts for such movements based on the sounds, adjusting such movements in response to such sounds which may also vary depending upon the user input and/or movements of the entire system, and the like. The present invention further relates to various processes for making various members, units, and parts of such systems, for synchronizing such with the sounds, and for making such controlled by such sounds.

Therefore, one objective of the present invention is to provide a pelvic relaxing system which automatically (or manually) plays preset sound during use of the system, in response to application of the user input thereto, based upon activation of its actuator member, and the like. A related objective of the present invention is to provide the system which automatically (or manually) play the sound as a preset part of such a system is engaged with the pelvic structure, e.g., when a tip of the system is in contact with the clitoris, when a preset part of the system is inserted into the internal cavity of such a structure, when the system is moved as a whole by the user, and the like.

Another objective of the present invention is to provide another pelvic relaxing system which moves different parts of its body member, effects different movements of such parts, and provides different stimuli to the same portion of the pelvic structure or different portions thereof depending on the sound provided thereto and/or retrieved thereby.

Another objective of the present invention is to provide another pelvic relaxing system which obtains sounds from various sources such as, e.g., an internal storage unit, external audio devices, external communication devices, internet, external storage media, and the like. A related objective is to provide the system which obtains audio signals from the sources and directly plays such signals to generate the sound or, in the alternative, which obtains control signals from such sources, forms or retrieves audio signals from the control signals, and plays such audio signals to generate the sound.

Another objective of the present invention is to provide another pelvic relaxing system which generates such sound based upon various user inputs, audio signals, and/or control signals supplied thereto from various sources. Thus, a related objective is to generate such sound in response to the user input supplied to the input and/or sensor units and/or various dynamic patterns thereof, based upon movements of the body parts and/or various dynamic features thereof, depending on disposition of various parts of the system, in response to movements of the system as a whole, depending upon the above internal and/or external audio and/or control signals, and the like.

Another objective of the present invention is to provide another pelvic relaxing system which generates such sounds depending upon various physiological variables measured in and around the pelvic structure.

Another objective of the present invention is to provide another pelvic relaxing system which generates such sound while effecting acoustic stimuli and delivering such stimuli to the portion of the pelvic structure. A related objective is to provide the system including an auxiliary driver unit which drives a preset part of the system based on the sound provided to and/or generated by the system.

Another objective of the present invention is to provide another pelvic relaxing system which generates the sound with only one of a content basis, a voice basis, and an action basis, generates such sound defining two of such bases, generates such sound having all of such bases, and so on, A related objective is to provide the system which generates a compound signal from multiple audio signals by superposing one of such basis of one audio signal onto another audio signal.

Another objective of the present invention is to provide another pelvic relaxing system which synchronizes an audio signal by adding the voice basis of one audio signal onto another audio signal, replacing one of such bases of one audio signal by a similar basis of another audio signal, altering at least one of such bases of one signal, and the like.

Another objective of the present invention is to provide another pelvic relaxing system which generates different movements of one or multiple parts of its body member based upon pre-recorded and/or real-time audio or control signals supplied thereto by an user and/or a third party, depending on such signals provided thereto through an external audio device, an external communication device or an internet, in response to compound and/of synthesized audio or control signals, and the like.

Another objective of the present invention is to provide another pelvic relaxing system which generates different sounds depending upon various criteria and/or conditions as enumerated in all of the above objectives.

Another objective of the present invention is to provide another pelvic relaxing system which generates such sound by playing a single or multiple audio signals for a preset period of time, e.g., by playing and repeating the same audio signal or the same set of such signals, by playing and repeating different audio signals or different sets thereof randomly or according to a preset order, by playing and repeating such audio signals in response to the user input or external pre-recorded or real-time control signals, and so on.

Various aspects and/or embodiments of various systems, methods, and/or processes of this invention will now be described, where such aspects and/or embodiments only represent different forms. Such systems, methods, and/or processes of this invention, however, may also be embodied in many other different forms and, therefore, should not be limited to the aspects and/or embodiments which are set forth herein. Rather, various exemplary aspects and/or embodiments described herein are provided so that this disclosure will be thorough and complete, and fully convey the scope of the present invention to one of ordinary skill in the art. It is to be understood that various movements and mechanisms therefor as well as various control algorithms of the prior art devices as described in the above Background of the Invention are to be incorporated herein in their entireties by reference.

In one aspect of the present invention, a pelvic relaxing system may provide tactile stimuli onto at least a portion of a pelvic structure while providing sound, where such a pelvic structure includes an entry and a wall, where the entry is arranged to define an orifice therethrough, and where the wall is arranged to include muscles and to define an internal cavity extending inwardly from the entry and bound by the muscles. Such a pelvic structure is to be referred to as the “standard pelvic structure” or simply the “pelvic structure” hereinafter for simplicity of illustration, unless otherwise specified.

In one exemplary embodiment of the present invention, a pelvic relaxing system may include at least one body member, at least one actuator member, and at least one control member. Such a body member may be arranged to include a first part for contacting the portion of the pelvic structure when engaged therewith and to include a second part for providing a grip to an user. Such a body member is to be referred to as the “body member of the first type” hereinafter for simplicity of illustration. The actuator member may be arranged to effect at least one movement of the first part for providing such stimuli onto the portion of the structure through the movement. Such an actuator member will now be referred to as the “actuator member of the first type” hereinafter for ease of illustration. The control member may be arranged to include at least one of at least one sensor unit and at least one input unit, to receive at least one user input defining at least one dynamic pattern with at least one of such units, and to effect the movement of the first part in response to the dynamic pattern of the user input. Such a control member is to be referred to as the “control member of the first type” hereinafter for ease of illustration.

In one example, the control member may also generate the sound during use of the system. In another example, the control member may also include at least one audio output unit which is capable of generating the sound when the user input is supplied to such at least one of the input and sensor unit. In another example, the control member may also include at least one audio output unit capable of generating the sound when the actuator member starts to provide such stimuli in response to the user input. In another example, such a control member may also include at least one main switch capable of moving or operating between at least one on-state and at least one off-state and then initiating and stopping operation of the actuator and control members, respectively, and include at least one audio output unit capable of generating the sound when the main switch is moved to or disposed in the on-state. In another example, the control member may also include at least one audio output unit capable of generating the sound when the first part is inserted into the cavity through the orifice. In another example, the control member may also include at least one audio output unit capable of generating the sound when the sensor unit detects movement of the body member.

In another exemplary embodiment of the present invention, a pelvic relaxing system may have at least one body member of the first type, at least one actuator member of the first type, and at least one control member of the first type. In one example, the control may also include at least one storage unit capable of storing at least one pre-recorded audio signal of the user and/or a third party, and then generate the sound by playing such an audio signal. In another example, the control member may also receive at least one pre-recorded audio signal of the user and/or a third party from an external audio device, and generate the sound by playing the audio signal. In another example, the control member may also receive at least one pre-recorded audio signal and/or real-time audio signal of a third party from an external communication device, and then generate the sound by playing the audio signal. In another example, the control member may also receive at least one pre-recorded audio signal and/or real-time audio signal from a third party through an internet, and generate the sound by playing such an audio signal. In another example, the control member may also receive at least one pre-recorded control signal of the user and/or a third party from an external audio device, and generate such sound based upon the control signal. In another example, the control member may also receive at least one pre-recorded control signal and/or real-time control signal of a third party by a communication device, and generate the sound based upon the control signal. In another example, the control member may also receive at least one pre-recorded control signal and/or real-time control signal of a third party by an internet, and generate the sound based upon such a control signal. In another example, the control member may also receive multiple pre-recorded audio signals and/or real-time audio signals of a third party and/or the user, generate a compound signal by combining at least two of such audio signals, and generate the sound by playing such a compound signal. In another example, the control member may also receive at least one pre-recorded audio signal defining a first content basis and a first voice basis and/or at least one real-time audio signal defining a second content basis and a second voice basis of the user and/or a third party, generate at least one synthesized signal by altering the content basis and/or voice basis of the signal, and generate the sound by playing the synthesized signal.

In another aspect of the present invention, a pelvic relaxing system may provide tactile stimuli onto at least a portion of the standard pelvic structure while providing sound in response to a control signal.

In one exemplary embodiment of the present invention, a pelvic relaxing system may also have at least one body member of the first type, at least one actuator member of the first type, and at least one control member which may be arranged to include at least one of a sensor unit and at least one input, to receive an user input defining at least one dynamic pattern, to effect the movement of the first part in response to the dynamic pattern of the user input, and to include at least one audio output unit capable of generating the sound. Such a control member is to be referred to as the “control member of the second type” hereinafter for ease of illustration.

In one example, the control member may also issue the control signal based upon the dynamic pattern of the user input supplied to the input unit by a hand of the user, thereby generating a distinct sound by the audio output unit based on the dynamic pattern. In another example, the control member may also issue the control signal based on the dynamic pattern of the user input supplied to the input unit through the pelvic structure of the user, thereby generating a distinct sound with the audio output unit based on the dynamic pattern. In another example, the control member may also issue the control signal based upon at least one dynamic feature such movement of the first part, thereby generating a distinct sound by the audio output unit based on its dynamic feature. In another example, the control member may also include a main switch capable of operating between an on-state and an off-state to turn on and off the actuator member, respectively, and issue the control signal based upon such state of the actuator member, thereby generating such sound with the audio output unit when the actuator member is moved to or disposed in the on-state. In another example, such a control member may also monitor insertion of the first part into the cavity through the opening of the pelvic structure, and issue the control signal based on detecting the insertion, thereby generating such sound by the audio output unit upon detecting such insertion. In another example, the control member may also issue the control signal based on the movement of the first part, thereby generating a distinct sound by the audio output unit based upon such movement. In another example, another control member may also monitor verbal command signal from the user, and issue the control signal based upon the command signal, thereby generating a distinct sound with the audio output unit based upon such a command signal. In another example, the control member may also receive at least one audio signal from an external audio device, and then issue the control signal based upon the audio signal, thereby generating a distinct sound by the audio output unit in response to the audio signal. In another example, the control member may also receive at least one audio signal from an external communication device, and issue the control signal based on the audio signal, thereby generating a distinct sound by the audio output unit in response to the audio signal. In another example, the control member may also receive the control signal from an external audio device, thereby generating a distinct sound by the audio output unit in response to such a control signal. In another example, the control member may also receive the control signal through an external communication device, thereby generating a distinct sound with the audio output unit as a response to the control signal. In another example, the control member may also monitor at least one physiological variable related to the pelvic structure, and then issue the control signal based upon the physiological variable, thereby generating a distinct sound by the audio output unit in response to the physiological variable.

In another exemplary embodiment of the present invention, such a pelvic relaxing system may have at least one body member of the first type, at least one actuator member, and at least one control member of the second type. Such an actuator member may be arranged to effect multiple movements of the first part for providing the stimuli onto the portion of the structure through the movements. The control member may also issue the control signal based on at least one of such movements of the first part as designated by such an user input, thereby generating different sounds by the audio output unit depending upon which of the movements is selected by the user input.

In another exemplary embodiment of the present invention, a pelvic relaxing system may have at least one body member of the first type, multiple actuator members, and at least one control member of the second type, where each of such actuator member may then be arranged to effect at least one movement of a single first part or multiple first parts for providing the stimuli to the portion of the pelvic structure through the movement. The control member may also issue the control signal based upon at least one of the actuator members designated by the user input, thereby generating different sounds by the audio output unit depending upon which of the actuator members is selected by the user input.

In another exemplary embodiment of the present invention, such a pelvic relaxing system may have at least one body member, at least one actuator member, and at least one control member of the second type. The body member may be arranged to include multiple first parts for contacting a single portion or multiple portions of the structure when engaged therewith and a second part for providing a grip to an user. The actuator member may be arranged to effect at least one movement of each of the first parts for providing such stimuli to the portion of the single structure or multiple structures through the movement. The control member may also issue the control signal based upon at least one of such first parts designated by the user input, thereby generating different sounds with the audio output unit depending upon which of the first parts is selected by the user input.

In another aspect of the present invention, another pelvic relaxing system may provide tactile stimuli to at least a portion of the standard pelvic structure while generating sound in response to an user input.

In one exemplary embodiment of the present invention, a pelvic relaxing system may include at least one body member of the first type, at least one actuator member of the first type, and at least one control member of the first type, where the control member may also generate the sound based on the user input.

In one example, the control member may also obtain such sound defining a content basis and a voice basis, and play the sound defining the content basis and the voice basis in response to the user input. In another example, another control member may obtain such sound defining an action basis but neither a content basis nor a voice basis, and play the sound with the action basis in response to the user input. In another example, the control member may also obtain the sound defining a background basis but neither a content basis nor a voice basis, and play the sound with the background basis in response to the user input. In another example, the control member may also obtain the sound with a content basis and/or voice basis, obtain a background basis, superpose the background basis to the sound, and play the superposed sound in response to the user input. In another example, the control member may also obtain the sound defining a background basis, obtain a content basis and/or a voice basis, superpose the voice basis and/or the content basis onto the sound, and play such superposed sound in response to such an user input. In another example, the control member may also obtain the sound having a content basis and/or a voice basis, obtain an action basis, superpose the action basis to the sound, and then play the superposed sound in response to the user input. In another example, the control member may also obtain the sound defining an action basis, obtain a content basis and/or a voice basis, superpose the voice basis and/or the content basis to such sound, and then play such a superposed sound as a response to the user input. In another example, the control member may also obtain the sound defining a background basis, obtain an action basis, superpose the action basis onto the sound, and then play the superposed sound in response to the user input. In another example, the control member may also obtain the sound which defines an action basis, obtain a background basis, superpose the action basis to the sound, and then play the superposed sound in response to the user input.

In another exemplary embodiment of the present invention, a pelvic relaxing system may also include at least one body member of the first type, at least one actuator member of the first type, and at least one control member of the first type. In one example, such a control member may also obtain a first content basis, obtain a second voice basis of a second person, synthesize a second sound defining the first content basis in terms of the second voice basis, and play the synthesized second sound in response to the user input. In another example, the control member may also obtain a first sound with a first content basis and a first voice basis of a first person, obtain a second voice basis of a second person, synthesize a second sound by replacing the first voice basis of the first sound by the second voice basis, and play the synthesized second sound in response to the user input. In another example, the control member may also obtain a first sound defining a first content basis and a first voice basis of a first person, obtain a second content basis of a second person, synthesize a second sound by replacing the first content basis of the first sound by the second content basis, and play the synthesized second sound as a response to the user input. In another example, the control member may further obtain a first sound defining a first content basis and a first voice basis of a first person, synthesize a second sound by altering at least a part of the first voice basis while maintaining such a first content basis, and play the synthesized second sound in response to the user input.

In another exemplary embodiment of the present invention, a pelvic relaxing system may have at least one body member of the first type, at least one actuator member of the first type, and at least one control member of the first type.

In one example, the control member may generate the sound in at least partial synchronization with such dynamic pattern of the user input supplied to the input unit by a hand of the user as well. In another example, the control member may also generate such sound in at least partial synchronization with the dynamic pattern of the user input supplied to the input unit through at least one portion of the pelvic structure of the user. In another example, the control member may also generate such sound in at least partial synchronization with at least one dynamic feature the movement of such a first part. In another example, the control member may also include a main switch capable of moving between an on-state and off-state to turn on and off the actuator member, respectively, and generate the sound in at least partial synchronization with the on-state of the actuator member. In another example, such a control member may also sense insertion of the first part into the pelvic cavity through its opening, and generate such sound in at least partial synchronization with the insertion. In another example, such a control member may also generate such sound in at least partial synchronization with such movement of the first part of the body member. In another example, the control member may also monitor verbal command signal from the user, and generate the sound in at least partial synchronization with such a command signal. In another example, the control member may also receive at least one audio signal from an external audio device, and then generate the sound in at least partial synchronization with the audio signal. In another example, the control member may receive at least one audio signal through an external communication device as well, and generate such sound in response to the audio signal. In another example, such a control member may also receive at least one control signal from an external audio device, and then generate such sound in at least partial synchronization with the control signal. In another example, the control member may also receive at least one control signal from an external communication device, and generate the sound in response to the control signal. In another example, the control member may also monitor at least one physiological variable inside the pelvic structure, and generate the sound in at least partial synchronization with the physiological variable.

In another exemplary embodiment of the present invention, a pelvic relaxing system may have at least one body member of the first type, at least one actuator member of the first type, and at least one control member of the first type which may also generate the sound while propagating acoustic waves of the sound toward the portion of the structure.

In another exemplary embodiment of the present invention, a pelvic relaxing system may have at least one body member of the first type, at least one actuator member of the first type, and at least one control member of the first type which may also generate the sound while controlling at least one dynamic feature of the movement of the first part based on such sound.

In another aspect of the present invention, another pelvic relaxing system may provide tactile stimuli to at least a portion of the standard pelvic structure and generating sound while controlling the stimuli through such sound.

In one exemplary embodiment of the present invention, a pelvic relaxing system may include at least one body member of the first type, at least one actuator member of the first type, and at least one control member of the first type.

In one example, the control member may further store multiple pre-recorded audio (or control) signals of the user and/or a third party, retrieve at least one of such signals, and then manipulate the actuator member and at least one dynamic feature of the movement of the first part effected thereby based upon the retrieved audio (or control) signal while generating the sound by playing the retrieved audio signal (or based on the control signal). In another example, the control member may also obtain an audio (or control) signal of the user, and manipulate the actuator member and at least one dynamic feature of the movement of the first part effected thereby based on the audio (or control) signal from the user while generating the sound by playing the audio signal (or based upon the control signal). In another example, the control member may receive a pre-recorded audio (or control) signal of the user and/or a third party through an external audio device, and manipulate the actuator member and at least one dynamic feature of the movement of the first part effected thereby based on the audio (or control) signal while generating such sound by playing the audio signal (or based upon the control signal). In another example, the control member may receive a real-time audio (or control) signal of a third party through an external communication device, and then manipulate the actuator member and at least one dynamic feature of the movement of the first part effected thereby based upon the audio (or control) signal while generating such sound by playing the audio signal (or based upon the control signal). In another example, the control member may receive at least one pre-recorded and/or real-time audio (or control) signal of a third party through an internet, and manipulate the actuator member and at least one dynamic feature of the movement of the first part effected thereby based on the audio (or control) signal while generating the sound by playing the audio signal (or based upon the control signal).

In another exemplary embodiment of the present invention, a pelvic relaxing system may have at least one body member of the first type, at least one actuator member of the first type, and at least one control member of the first type which may also generate such sound while propagating acoustic waves of the sound to the portion of the pelvic structure, thereby providing such stimuli to the portion not only by the movement of the first part but also by the acoustic waves.

In another exemplary embodiment of the present invention, a pelvic relaxing system may have at least one body member of the first type, at least one actuator member of the first type, at least one control member of the first type which may also generate the sound in response to the user input, and at least one auxiliary driver unit. In one example, the auxiliary driver unit may be arranged to effect at least one another movement of the first part based upon the sound. In another example, the auxiliary driver unit may be arranged to effect at least one another movement of the first part based upon such sound and to superpose such another movement of the first part onto such movement of the first part effected by the actuator member.

In another exemplary embodiment of the present invention, a pelvic relaxing system may have at least one body member, at least one actuator member, at least one control member of the first type, and at least one auxiliary driver unit. The body member may be arranged to include multiple first parts capable of contacting the portion of such a structure when engaged therewith and a second part for providing a grip to an user. The actuator member may be arranged to effect at least one movement of at least one of the first parts for providing the stimuli to the portion of the structure by such movement. The control member may also be arranged to generate such sound in response to the user input. The auxiliary driver unit may be arranged to effect at least one another movement of at least another of the first parts based upon the sound. Whereby such a system may be arranged to provide multiple stimuli to multiple the portions by such at least one of the first parts as well as by such at least another of the first parts.

In another aspect of the present invention, another pelvic relaxing system may provide tactile stimuli to at least a portion of such a standard pelvic structure while providing sound during a preset period of time.

In one exemplary embodiment of the present invention, a pelvic relaxing system may include at least one body member of the first type, at least one actuator member of the first type, and at least one control member of the first type.

In one example, the control member may also obtain an audio signal, and then generate such sound by repeating the audio signal during the period of time in response to the user input. In another example, the control member may also obtain a set of multiple audio signals, and generate such sound by repeating the set of such audio signals during the period of time as a response to the user input. In another example, the control member may further obtain at least two audio signals, and then generate the sound by repeating the audio signals during the period of time in a preset (or random) order which may be determined by the user input. In another example, the control member may also obtain a set of multiple audio signals, and generate such sound by repeating the set of such audio signals during the period of time in a preset (or random) order determined by the user input. In another example, such a control member may also obtain multiple audio signals, receive a pre-recorded control signal from at least one of the user an a third party, and generate such sound in an order determined by the control signal during the period of time. In another example, the control member may also obtain multiple audio signals, receive a real-time control signal from a third party, and generate the sound in an order which is determined by the control signal during the period of time. In another example, the control member may also obtain multiple audio signals, receive at least one pre-recorded control signal and/or a real-time control signal from a third party, and generate the sound in an order at least partially determined by the control signal during the period of time. In another example, the control member may also obtain multiple audio signals each defining at least one of a background basis, an action basis, a voice basis, and a content basis, categorize the audio signals into at least two groups based on at least one of the bases, and generate such sound by playing the audio signals of a preset group selected by the user input and repeating such audio signals when necessary during the period of time. In another example, the control member may also obtain multiple audio signals each defining at least one of an action basis, a voice basis, a background basis, and a content basis, categorize the audio signals into at least two groups based on at least one of the bases, and generate such sound by playing such audio signals of different groups one at a time as determined by the user input and/or by the movement of the part and repeating the audio signals when necessary during the period of time. In another example, the control member may also obtain multiple audio signals, monitor the dynamic pattern of the user input applied to the input unit by a hand of the user, and generate the sound in an order at least partially determined by the dynamic pattern during the period of time. In another example, the control member may also obtain multiple audio signals, monitor the dynamic pattern of the user input applied to the sensor unit through the pelvic structure of the user, and generate the sound in an order at least partially determined by the dynamic pattern during the period of time. In another example, the control member may obtain multiple audio signals, monitor at least one dynamic feature of the movement of the part, and then generate the sound in an order at least partially determined by the dynamic feature during the period of time.

In another exemplary embodiment of the present invention, such a pelvic relaxing system may have at least one body member of the first type, at least one control member of the first type, as well as multiple actuator members each of which may be arranged to effect at least one movement of the first part for providing the stimuli onto the portion of the structure through the movement. The control member may also obtain multiple audio signals, and then generate the sound by playing different audio signals and repeating the audio signals when necessary depending upon at least one of the actuator members designated by the user input during the period of time.

In another exemplary embodiment of the present invention, a pelvic relaxing system may have at least one body member of the first type, at least one actuator member of the first type, and at least one control member of the first type which may also monitor disposition of the first part, obtain at least one audio signal, and generate the sound by playing the audio signal during the period of time and also during detecting the insertion of the first part inside the pelvic structure.

In another exemplary embodiment of the present invention, a pelvic relaxing system may have at least one body member of the first type, at least one actuator member of the first type, and at least one control member of the first type. In one example, the control member may also sense the dynamic pattern of the user input, obtain multiple audio signals, and then generate the sound system by playing different audio signals based on the dynamic pattern during the period of time. In another example, the control member may also monitor at least one dynamic feature of such movement, obtain multiple audio signals, and then generate the sound by playing different audio signals based on the dynamic feature during the period of time.

Embodiments of such apparatus aspects of the present invention may include one or more of the following features.

The pelvic relaxing system may be arranged to allow its user to reach and manipulate the input unit (or the switch) by at least one finger thereof while simultaneously holding and/or manipulating the second part with a hand thereof during use of the system without having to move the hand relative to the second part. The system may also be arranged to allow the user to provide the user input through contacting and abutting the sensor unit with at least a portion of the pelvic structure while holding and manipulating the second part with a hand thereof during use of the system without having to move the finger thereof.

The input unit (or switch) may receive the user input with at least a substantially identical area thereof to actuate the actuator member, effect such movements, provide the stimuli to the portion, and the like. The input unit (or switch) and/or the sensor unit may move or operate between the states at least substantially continuously or intermittently. The input unit (or switch) and/or sensor unit may be a joystick, a touch pad, a track ball, and other switches which may be arranged to move horizontally and/or vertically while operating between the states. Such a joystick, touch pad, and/or track ball may be arranged to move horizontally and/or vertically as a whole in addition to the operating movements as well. The sensor unit may be incorporated into the first part of the body member which may be a head, a trunk, and/or a base thereof. The sensor unit may be incorporated into the second part of the body member when desirable. Such a control member may also include conventional on/off switches, conventional speed control switches, and the like.

Such a portion of the pelvic structure may correspond to a clitoris of the entry, a G spot on the wall, other portions of the wall, and the like. The part of the body member may also be designed as a clitoral stimulator, as a G spot stimulator, and the like, where such a part may be defined in a head, a trunk, and/or a base of the body member. The body member may include multiple parts which may be identical, disposed close to each other, disposed apart from each other, and the like.

The dynamic pattern of the user input may include at least one of its temporal pattern and its spatial pattern, where the temporal pattern may include at least one of a duration of the user input, its frequency, and its temporal sequence, and where the spatial pattern may include at least one of an amplitude of the user input (or displacement), its direction, and its type which may be at least one of vibration, horizontal translation, vertical translation, transverse translation, angular rotation, rotation about a center of rotation and/or an axis of rotation defined in the body member, swivelling, tapping, deformation, and so on. The dynamic pattern may be at least one of a frequency of the user input, its temporal rate of change (or acceleration), its displacement (or its integral over time) caused thereby, and a compound value obtained by at least one of mathematical manipulation of at least one thereof. The user input may also be a presence and absence of a contact between the user and input unit (or switch) and/or sensor unit regardless of the force accompanying the contact.

The dynamic feature of the movement may include at least one of its temporal feature and its spatial feature, where the temporal feature may include at least one of a duration of the movement, its frequency, and its temporal sequence, and where the spatial feature may include at least one of an amplitude of the movement (or displacement), its direction, and its type which may be at least one of vibration, horizontal translation, vertical translation, transverse translation, angular rotation, rotation about a center of rotation and/or an axis of rotation defined in the body member, swivelling, tapping, deformation, and the like. The temporal feature may include one or multiple movements of a preset part or different parts of the body member which may be arranged in the sequence which may be a preset sequence or a sequence which is at least partially determined by the user input. The spatial feature may include one or multiple movements of a preset part or different parts of the body member while contacting a preset portion or different portions of the pelvic structure.

In another aspect of the present invention, a method may provide tactile stimuli effected by at least one part of a body member of a pelvic relaxing system to at least a portion of the standard pelvic structure while playing sound in response to operation of the system.

In one embodiment of this aspect of the present invention, a method may include the steps of: engaging the body member with the pelvic structure; supplying at least one user input to the system; effecting at least one movement of such a body part in response to the user input; and generating the sound in response to at least one of the engaging, supplying, and effecting. Such generating may be replaced by the step of: synchronizing such sound with at least one of the engaging, supplying, and effecting.

In another embodiment of this aspect of the present invention, a method may include the steps of: engaging the body member with the pelvic structure; supplying the system with at least one user input; monitoring movement of the system; and generating the sound when a displacement, a velocity, and/or an acceleration of movement of the entire system exceeds a preset value. Such generating may also be replaced by the step of: synchronizing the sound with a displacement, a velocity, and/or an acceleration of movement of the entire system.

In another embodiment of this aspect of the present invention, a method may include the steps of: supplying at least one user input to the system; effecting at least one movement of the body part in response to the user input; inserting at least a part of the system into the cavity; and generating such sound in response to the inserting. Such generating may be replaced by the step of: synchronizing the sound with the inserting.

In another embodiment of this aspect of the present invention, a method may include the steps of: initiating (or turning on) the system; engaging the body member with the pelvic structure; supplying at least one user input to the system; effecting at least one movement of the body part in response to the user input; and generating the sound in response to initiating. Such generating may be replaced by the step of: synchronizing the sound with initiating.

In another aspect of the present invention, a method may provide tactile stimuli effected by at least one part of a body member of a pelvic relaxing system to at least a portion of the standard pelvic structure while generating sound from at least one audio signal.

In one embodiment of this aspect of the present invention, a method may include the steps of: engaging the body member with the pelvic structure; supplying at least one user input to the system; effecting at least one movement of the body part in response to the user input; storing multiple pre-recorded audio signals in the system; obtaining at least preset audio signal from the system; and then playing the obtained audio signal in response to at least one of the engaging, supplying, and effecting, thereby generating the sound. The storing and obtaining may be replaced by the step of: obtaining at least one pre-recorded audio signal from an user and/or a third party through an external audio device or may also be replaced by the step of: obtaining at least one of a pre-recorded audio signal and real-time audio signal of a third party through an external communication device. The storing and obtaining may instead be replaced by the step of: obtaining at least one of a pre-recorded audio signal and real-time audio signal of a third party through an internet. In addition, such playing may be replaced by the step of: synchronizing the sound which is based upon the obtained audio signal with at least one of the engaging, supplying, and effecting, thereby generating the sound.

In another embodiment of this aspect of the present invention, a method may include the steps of: engaging the body member with such a structure; supplying at least one user input to the system; effecting at least one movement of the body part as a response to the user input; storing multiple pre-recorded control signals in the system; obtaining at least preset control signal from the system; and generating the sound based on the obtained control signal in response to at least one of the engaging, supplying, and effecting. Such storing and obtaining may be replaced by the step of: obtaining at least one pre-recorded control signal from an user and/or a third party through an external audio device. Such storing and obtaining may be replaced by the step of: obtaining at least one of a pre-recorded control signal and real-time control signal from a third party through an external communication device. Such storing and obtaining may be replaced by the step of: obtaining at least one of a pre-recorded control signal and real-time control signal from a third party through an internet. In addition, the above generating may be replaced by the step of: synchronizing the sound which is based on the obtained control signal with at least one of the engaging, supplying, and effecting.

In another aspect of the present invention, a method may provide tactile stimuli effected by at least one part of a body member of a pelvic relaxing system to at least a portion of the standard pelvic structure while playing sound in response to at least one control signal.

In one embodiment of this aspect of the present invention, a method may include the steps of: engaging the body member with the pelvic structure; supplying at least one user input to the system by a hand of an user; issuing the control signal based upon at least one dynamic pattern of the user input; effecting at least one movement of such a body part in response to the control signal; and then generating the sound in response to the control signal. Such supplying may be replaced by the step of: supplying at least one user input to the system through the pelvic structure of an user. In addition, such generating may be replaced by the step of: synchronizing the sound with the control signal.

In another embodiment of this aspect of the present invention, a method may include the steps of: engaging the body member with the pelvic structure; providing at least one user input to an input (or sensor) unit of the system; issuing the control signal based on movement of the input (or sensor) unit caused thereby; effecting at least one movement of such a body part in response to the control signal; and generating the sound in response to the control signal. Such generating may be replaced by the step of: synchronizing the sound with the control signal.

In another embodiment of this aspect of the present invention, a method may include the steps of: configuring the system to effect multiple movements of the body part; supplying at least one user input to the system' effecting at least one of the movements of the body part based on the user input; issuing the control signal based on the effected movement; and generating the sound in response to the control signal. Such generating may be replaced by the step of: synchronizing the sound with the control signal.

In another embodiment of this aspect of the present invention, a method may include the steps of: configuring the system to define multiple the parts on the body member; supplying at least one user input to the system; effecting movement of at least one of the body parts based upon the user input; issuing the control signal based on the part effected by the movement; and generating such sound in response to the control signal. Such generating may be replaced by the step of: synchronizing such sound with the control signal.

In another embodiment of this aspect of the present invention, a method may include the steps of: configuring the system to define multiple actuator members each of which is capable of effecting movement of the body part; supplying at least one user input to the system; effecting movement of the body part by at least one of the actuator members based on the user input; issuing the control signal based on the actuator member effecting the movement; and generating the sound in response to the control signal. Such generating may be replaced by the step of: synchronizing the sound with such a control signal.

In another embodiment of this aspect of the present invention, a method may include the steps of: engaging the part of the body member with the pelvic structure; supplying at least one user input and at least one verbal command signal to the system; effecting movement of the body part based on the user input; issuing the control signal based upon the verbal command signal; and then generating the sound in response to such a control signal. The above generating may be replaced by the step of: synchronizing the sound with the control signal.

In another embodiment of this aspect of the present invention, a method may include the steps of: engaging the part of the body member with the pelvic structure; supplying at least one user input to the system; effecting movement of the body part based on the user input; obtaining at least one audio signal through an external audio device, an external communication device, and/or an internet; issuing the control signal based on the obtaining; and generating the sound in response to the control signal. Such generating may be replaced by the step of: synchronizing the sound with the control signal.

In another embodiment of this aspect of the present invention, a method may include the steps of: engaging the part of the body member with the pelvic structure; supplying at least one user input to the system; effecting movement of the body part based on the user input; obtaining the control signal through at least one of an internet, an external audio device, and an external communication device; issuing the control signal based on the obtaining; and generating the sound in response to the control signal. Such generating may be replaced by the step of: synchronizing the sound with such a control signal.

In another embodiment of this aspect of the present invention, a method may include the steps of: engaging the part of the body member with the pelvic structure; supplying at least one user input to the system; effecting movement of the body part based upon the user input; issuing the control signal based on the engaging; and generating the sound in response to the control signal. Such generating may be replaced by the step of: synchronizing the sound with the control signal.

In another embodiment of this aspect of the present invention, a method may include the steps of: supplying at least one user input to the system; effecting movement of the body part based on the user input; moving the entire system with respect to such a structure; issuing the control signal based on the moving; and generating the sound in response to the control signal. Such generating may be replaced by the step of: synchronizing the sound with the control signal.

In another embodiment of this aspect of the present invention, a method may include the steps of: engaging the part of the body member with the pelvic structure; effecting movement of the body part based on the user input; monitoring at least one physiological variable of such a structure; issuing the control signal based on the physiological variable; and generating the sound in response to such a control signal. Such generating may be replaced by the step of: synchronizing the sound with such a control signal.

In another aspect of the present invention, a method may provide tactile stimuli effected by at least one part of a body member of a pelvic relaxing system to at least a portion of the standard pelvic structure while playing sound in response to an user input.

In one embodiment of this aspect of the present invention, a method may include the steps of: engaging the body member with the pelvic structure; supplying at least one user input to the system; effecting at least one movement of the body part in response to the user input; generating the sound in response to the user input; and then effecting acoustic stimuli to the portion by directing the sound thereto.

In another embodiment of this aspect of the present invention, a method may include the steps of: engaging the body member with the pelvic structure; supplying the system with at least one user input; effecting at least one movement of the body part in response to the user input; generating the sound in response to the user input; and effecting acoustic stimuli to the portion by directing the sound thereto.

In another embodiment of this aspect of the present invention, a method may include the steps of: configuring the system to define multiple the parts on the body member; supplying at least one user input to the system; effecting at least one movement of a first of the body parts as a response to the user input; generating the sound in response to the user input and/or movement; and effecting at least another movement of a second of the body parts caused by acoustic stimuli in response thereto.

In another aspect of the present invention, a method may provide tactile stimuli effected by at least one part of a body member of a pelvic relaxing system to at least a portion of the standard pelvic structure while playing a synthesized sound.

In one embodiment of this aspect of the present invention, a method may include the steps of: supplying at least one user input to the system; effecting at least one movement of the body part as a response to the user input; obtaining a first content basis; selecting a second voice basis; and then generating such a synthesized sound by defining the first content basis in terms of the second voice basis during at least a portion of the effecting.

In another embodiment of this aspect of the present invention, a method may include the steps of: supplying at least one user input to the system; effecting at least one movement of the body part in response to the user input; obtaining a first sound with a first content basis and a first voice basis of a first person; selecting a second voice basis a second person; and then generating the synthesized sound by replacing the first voice basis of the first sound with the second voice basis during at least a portion of the effecting.

In another embodiment of this aspect of the present invention, a method may include the steps of: supplying at least one user input to the system; effecting at least one movement of the body part in response to the user input; obtaining a first sound with a first voice basis of a first person; and then generating the synthesized sound through altering the first voice basis during at least a portion of the effecting.

In another aspect of the present invention, a method may be arranged to play sound as well as to provide tactile stimuli effected by at least one part of a body member of a pelvic relaxing system to at least a portion of the standard pelvic structure while synchronizing the stimuli with such sound.

In one embodiment of this aspect of the present invention, a method may include the steps of: engaging the body member with the pelvic structure; obtaining at least pre-recorded audio (or control) signal; and then generating the sound through playing the audio signal (or based on the control signal) while effecting at least one movement of the body part in response to the sound (or signal).

In another embodiment of this aspect of the present invention, a method may include the steps of: engaging the body member with the pelvic structure; obtaining at least one of a pre-recorded and real-time audio (or control) signal; and generating the sound through playing the audio signal (or based on the control signal) while effecting at least one movement of the body part in response to the sound (or signal).

In another embodiment of this aspect of the present invention, a method may include the steps of: engaging the body member with the pelvic structure; obtaining at least one of a pre-recorded and real-time audio (or control) signal through an external audio device, an external communication device, and/or an internet; and generating the sound through playing the audio signal (or based on the control signal) while effecting at least one movement of the body part in response to the sound (or signal).

In another embodiment of this aspect of the present invention, a method may include the steps of: engaging the body member with the pelvic structure; obtaining at least two of a pre-recorded and real-time audio (or control) signal; generating a compound audio (or control) signal from at least two of the signals; and then generating the sound by playing the compound audio signal (or based upon the compound control signal) while effecting at least one movement of the body part as a response to the sound (or compound signal).

In another embodiment of this aspect of the present invention, a method may include the steps of: engaging the body member with the pelvic structure; obtaining at least two of a pre-recorded and real-time audio signals each of which may define at least one of a content basis, a voice basis, and an action basis; generating a synthesized audio signal from at least two of the signals by varying at least one of the bases; and generating the sound through playing the synthesized audio signal while effecting at least one movement of the body part in response to the sound (or synthesized signal).

In another aspect of the present invention, a method may provide tactile stimuli effected by at least one part of a body member of a pelvic relaxing system to at least a portion of the standard pelvic structure while playing sound for a preset period of time.

In one embodiment of this aspect of the present invention, a method may include the steps of: engaging the body member with the pelvic structure; supplying at least one user input to the system; effecting at least one movement of the body part in response to the user input; obtaining at least one audio signal; and generating such sound by playing the audio signal and repeating the playing when necessary in response to the user input during the period of time. Such obtaining and generating may be replaced by the steps of: obtaining multiple audio signals; categorizing the audio signals into multiple groups; and generating the sound by playing the audio signal from at least one of the groups and then repeating the playing when necessary in response to the user input during the period of time. Such obtaining and generating may be replaced by the steps of: obtaining multiple audio signals; and then generating the sound by playing the audio signals based on a preset order and repeating the playing when necessary in response to the user input during the period of time. The obtaining and generating may further be replaced by the steps of: obtaining multiple audio signals; receiving an external control signal for determining a preset order by an external audio device, an external communication device, and/or an internet; and generating the sound by playing the audio signals based on the order and then repeating the playing when necessary in response to the user input during the period of time.

In another embodiment of this aspect of the present invention, a method may include the steps of: engaging the body member with the pelvic structure; supplying the system with at least one user input; effecting at least one movement of the body part in response to the user input; obtaining multiple audio signals; and then generating the sound by playing the audio signals in an order at least partially determined by the user input during the period of time. The generating may be replaced by the step of: generating the sound by playing the audio signals in an order at least partially determined by at least one dynamic pattern of the user input during the period of time. Such generating may be replaced by the step of: generating the sound by playing the audio signals in an order at least partially determined by the movement of the body part during the period of time or by the step of: generating the sound by playing the audio signals in an order at least partially determined by at least one dynamic feature of the movement of the body part during the period of time. The generating may further be replaced by the step of: generating the sound by playing the audio signals in an order at least partially determined by movement of the system as a whole during the period of time or by the step of: generating the sound by playing the audio signals in an order at least partially determined by at least one dynamic feature of movement of the system as a whole during the period of time. The generating may be replaced by the step of: generating the sound by playing the audio signals in an order at least partially determined by disposition of the system with respect to the pelvic structure during the period of time or by the step of: generating such sound by playing the audio signals in an order at least partially determined by at least one verbal command signal from the user during the period of time.

Embodiments of the above method aspects of the present invention may include one or more of the following features.

The effecting may include at least one of the steps of: effecting different movements defining different dynamic features; and changing at least one configuration of the body member which may be a length of the body member, its diameter, its curvature, and/or its surface texture. Such effecting may also include at least one of the steps of: controlling at least one of temporal feature and spatial feature of the movement; controlling at least one of a duration of the movement, its frequency, and its temporal sequence; controlling at least one of an amplitude of the movement (or its displacement), its direction, and its type; and selecting the movement from horizontal or vertical translation, transverse translation, vibration, angular rotation, rotation about a center of rotation or an axis of rotation defined in the body member, swivelling, tapping, deformation, and the like. The effecting may include the step of: effecting one or multiple movements of a preset part (or different parts) of the body member which may be arranged in the sequence which may in turn be a preset sequence or a sequence which may be at least partially determined by such an user input. The effecting may include the step of: effecting one or multiple movements of a preset part (or different parts) of the body member while contacting a preset portion (or different portions) of the pelvic structure. The contacting may also include the step of: engaging the part with one of a clitoris of the entry, a G spot on the wall, other portions of the wall, and the like.

The supplying may also include at least one of the steps of: supplying the user inputs without moving a hand of an user from the body member; supplying the user inputs without changing a grip of the body member; and supplying the user inputs by at least one finger of an user while holding a grip of the body member with the finger. The supplying may include at least one of the steps of: continuing the effecting only during a period of the supplying; and terminating the effecting in a preset period of terminating the supplying. The supplying may include one of the steps of: effecting only the movement intended by the user input; and superposing the movement intended by the user input onto a default movement. The supplying may be replaced by the step of: supplying different user inputs through the input unit, thereby changing a configuration of the body member without manually replacing the body member. The supplying may include at least one of the steps of: controlling at least one of temporal pattern and spatial pattern of the user input; controlling at least one of a duration of the user input, its frequency, and its temporal sequence; controlling at least one of an amplitude of the user input (or its displacement), its direction, and its type; and selecting the user input from at least one of vibration, horizontal or vertical translation, transverse translation, angular rotation, rotation about a center or an axis of rotation which may be defined in the body member, swivelling, tapping, deformation, and the like. The supplying may have the step of: controlling at least one of a frequency of the user input, its temporal rate of change (or acceleration), its displacement (or its integral over time) caused thereby, and a compound value obtained by a mathematical manipulation of at least one thereof. The supplying may further include the step of: initiating and terminating contact between the user and the input unit (or switch) regardless of the force accompanying the contact.

Various product-by-process claims may be constructed by modifying the foregoing preambles or their modifications of the above system and/or method claims and by appending thereto the above bodies or their modifications of the above system and/or method claims. Such process claims may be arranged to include one or more of the aforementioned features of the above system and/or method claims of the present invention.

As used herein, both of the terms “input unit” and “sensor unit” refer to those units of various pelvic relaxing systems capable of receiving user inputs and/or monitoring dynamic pattern thereof. However, the “input unit” and “sensor unit” are to be differentiated as follows within the scope of the present invention. First of all, the “input unit” is generally disposed in a part of a body member of such a system which is intended to not be inserted into a cavity of a pelvic structure, whereas the “sensor unit” is preferably disposed in such an insertable part of the body member. Therefore, the “input unit” mainly receives the user inputs through a hand or finger of an user, while the “sensor unit” generally receives the user inputs through various portions of the pelvic structure.

The terms “proximal” and “distal” will be used in a relative context. Throughout this invention, the term “proximal” is to be used to denote a direction toward a head of a body member of a system, while the term “distal” is to be used to denote an opposite direction toward an end of a handle of such a system. Accordingly, a “proximal” end and a “distal” end may be defined with respect to an entire pelvic relaxing system or with respect to a specific member or unit thereof.

The terms “input unit” and “sensor unit” generally refer to identical or similar articles capable of monitoring various dynamic patterns of various user inputs applied thereto. Throughout this invention, however, the “input units” represent such articles incorporated into a handle part (or a second unit) of a body member of a pelvic relaxing system, while the “sensor units” denote such articles incorporated into an insertable part (or a first unit) of such a body member. Accordingly, any articles which may be used as the “input unit” may also be used as the “sensor unit” unless otherwise specified. In addition, the “input unit” may also be disposed in the insertable part of the body member, while the “sensor unit” may also be disposed in the handle part thereof when desired.

As used herein, a “dynamic pattern” refers to a temporal pattern as well as a spatial pattern of an user input and/or of a sensing signal each of which is generated by an input unit and/or a sensor unit of a control member in response to the user input, while a “dynamic feature” refers to a temporal feature as well as a spatial feature of movement of a single part or multiple parts of a body member.

A “pelvic structure” means an anatomical structure including an entry and a wall, where such an entry forms an orifice therethrough, while the wall includes muscles and defines an internal cavity extending inwardly from the entry and bound by the muscles. Such a pelvic structure will be referred to as the “standard pelvic structure” or simply the “pelvic structure” hereinafter for ease of illustration, unless otherwise specified.

Unless otherwise defined in the following specification, all technical and scientific terms used herein have the same meaning as commonly understood by one of ordinary skill in the art to which the present invention belongs. Although the methods or materials equivalent or similar to those described herein can be used in the practice or in the testing of the present invention, the suitable methods and materials are described below. All publications, patent applications, patents, and/or other references mentioned herein (particularly those enumerated in the above Background section) are incorporated by reference in their entirety. In case of any conflict, the present specification, including definitions, will control. In addition, the materials, methods, and examples are illustrative only and not intended to be limiting.

Other features and advantages of the present invention will be apparent from the following detailed description, and from the claims.

### BRIEF DESCRIPTION OF THE DRAWING

FIGS. 1A to 1D are schematic cross-sectional diagrams of exemplary pelvic relaxing systems with audio input and output units disposed in different parts according to the present invention; and

FIG. 2 is a schematic diagram of various functional members and units of an exemplary pelvic relaxing system according to the present invention.

### DETAILED DESCRIPTION OF THE PREFERRED EMBODIMENTS

The present invention generally relates to pelvic relaxing systems capable of generating sound of an user or third party or capable of being manipulated by the sound. More particularly, the present invention relates to pelvic relaxing systems for acquiring real-time and/or pre-recorded sounds from a third party and/or an user and playing the sounds during use and/or in response to an user input, for synchronizing such sounds with movements of various parts of the systems and/or operations of the systems, for playing the sounds synthesized from multiple audio signals each defining different voice bases and/or content bases, and so on. The present invention also relates to pelvic relaxing systems of which operations may be controlled at least in part by such sounds so that movements of various parts and/or operations thereof may be controlled by and/or synchronized with the sounds which are provided thereto by audio and/or communication devices.

The present invention also relates to various methods of obtaining such sounds from various sources internal or external to such systems, playing such sounds in response to various operations of the systems, playing one or more of such sounds depending upon types of the movements of the parts of the system, synchronizing such sounds with various movements of such parts, types of the movements of such parts, and/or parts of the system recruited for such movements, selecting one or more parts of the system for such movements designated by the user input, and so on. The present invention also relates to various methods of manipulating such movements based upon such sounds, selecting one or multiple parts for such movements based on the sounds, adjusting such movements in response to such sounds which may also vary depending upon the user input and/or movements of the entire system, and the like. The present invention further relates to various processes for making various members, units, and parts of such systems, for synchronizing such with the sounds, and for making such controlled by such sounds.

Various aspects and/or embodiments of various systems, methods, and/or processes of this invention will now be described more particularly with reference to the accompanying drawings and text, where such aspects and/or embodiments thereof only represent different forms. Such systems, methods, and/or processes of this invention, however, may also be embodied in many other different forms and, accordingly, should not be limited to such aspects and/or embodiments which are set forth herein. Rather, various exemplary aspects and/or embodiments described herein are provided so that this disclosure will be thorough and complete, and fully convey the scope of the present invention to one of ordinary skill in the relevant art.

Unless otherwise specified, it is to be understood that various members, units, elements, and parts of various systems of the present invention are not typically drawn to scales and/or proportions for ease of illustration. It is also to be understood that such members, units, elements, and/or parts of various systems of this invention designated by the same numerals may typically represent the same, similar, and/or functionally equivalent members, units, elements, and/or parts thereof, respectively.

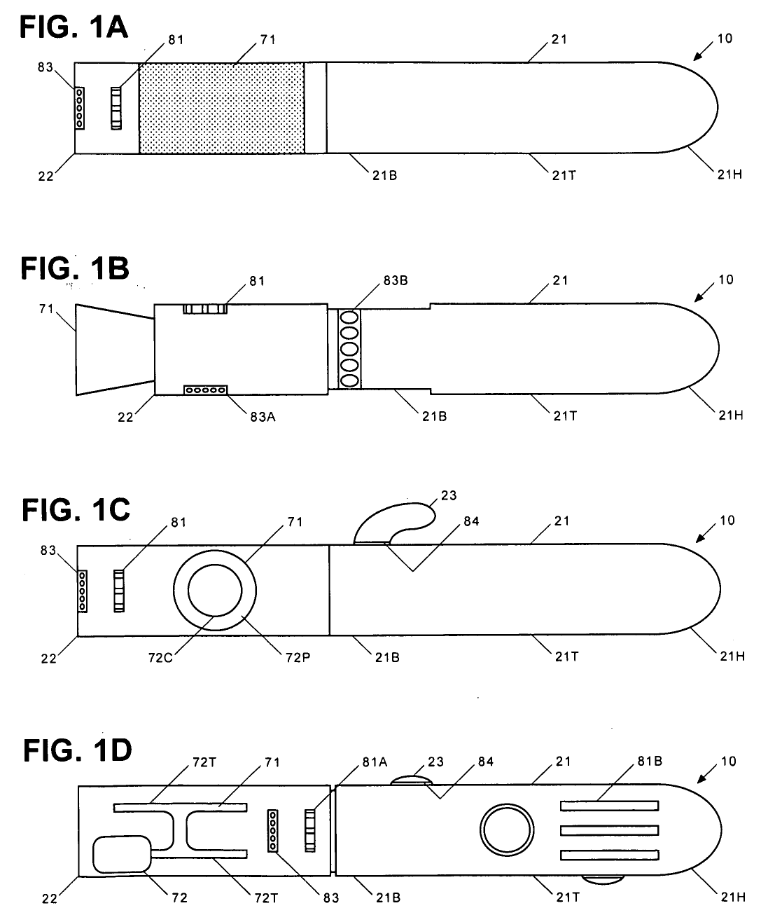

In one aspect of the present invention, pelvic relaxing systems may be provided with various members and units for effecting various movements and providing various stimuli to a portion and/or multiple portions of such a structure while generating various sounds with audio output units thereof. FIGS. 1A to 1D show schematic cross-sectional diagrams of exemplary pelvic relaxing systems with audio input and output units disposed in different parts according to the present invention.

In one exemplary embodiment of this aspect of the invention and as described in FIG. 1A, an exemplary pelvic relaxing system 10 includes a body member 20 with a first unit 21 and a second unit 22, an actuator member incorporated in the body member 20 (thus not shown in this figure), a control member with an input unit 71 and other units incorporated into the body member 20 (thus not included in this figure), at least one audio input unit 81, and at least one audio output unit 83. The first unit 21 is typically elongated and arranged to be inserted into the pelvic cavity during use, while the second unit 22 is distally coupled to the first unit 21 and arranged to form a grip for an user during use. The first unit 21 is divided into a head 21H, a trunk 21T, and a base 21B. The head 21H is formed in a proximal end of the body member 20 and rounded to facilitate insertion of the body member 20 into the pelvic cavity, and the trunk 21T is connected distally to the head 21 H and terminates in the base 21B which then couples to the second unit 22. In general, the head 21H, trunk 21T, and base 21B are fixedly or movably coupled to each other and form an unitary body member 20. In the alternative, the head 21H, trunk 21T, and base 21B may be arranged to form an unitary first unit 21. As far as the body member 20 may generate one or more desirable movements thereof, detailed construction and coupling modes of such parts 21H, 21T, 21B may not be generally material to the scope of the present invention. The second unit 22 is also elongated and has a shape and size to form the grip for the user, whether the user grabs the second unit 22 with his or her thumb pointing proximally, distally, and/or laterally. The second unit 22 may be movably or fixedly coupled to each other and form an unitary body member 20. Alternatively, the first and second units 21, 22 may instead define an unitary body member 20. As far as the body member 20 may generate one or more desirable movements, detailed construction and/or coupling modes of such first and second units 21, 22 are neither material to the scope of the present invention. As will be described below, the pelvic relaxing system 10 also includes other members and units for normal operation. When the first and/or second units 21, 22 may form cavities therein, such members and/or units may be incorporated into one or both of such units 21, 22. Alternatively, at least one of such members and units may be exposed through surfaces of such units 21, 22.

The input unit 71 is disposed around the second unit 22 and exposed to provide direct access to the user. The input unit 71 of this embodiment is arranged to receive an user input through at least a substantial area thereof. The input unit 71 is further disposed in a center part of the second unit 22, while defining vacant areas between itself 71 and a proximal end and a distal end of the second unit 22. Therefore, such an input unit 71 shares an area of the second unit 22 which is used to define the grip for the user. As will be explained in greater detail below, the input unit 71 is preferably arranged to monitor various dynamic patterns of mechanical or electrical contact between the user and/or force applied thereto by the user and to generate sensing signals based upon such dynamic patterns. More specifically, such an input unit 71 may generate the sensing signals of which amplitudes, durations or other temporal and/or spatial patterns may be determined by the contact and/or force for grabbing and holding the second unit 22. Thus, the user may control various features of pelvic relaxing operation while grabbing and holding the second unit 22 during use and without having to change the grip of the second unit 22, without having to move his or her hand in order to reach the input unit 71 while holding or grabbing the second unit 22, and the like.

The control member further includes the audio input unit 81 and audio output unit 83, where the main function of the audio input unit 81 is to acquire audio and/or control signals from various sources and where the main function of the audio output unit 83 is to generate sound based on such acquired audio and/or control signals. The audio input unit 81 may generally be fabricated of any conventional articles which are capable of converting acoustic waves into electrical and/or optical signals such as, e.g., microphones. The audio input unit 83 may be arranged as well to acquire electrical and/or optical audio and/or control signals from various conventional external devices such as, e.g., audio devices, visual devices with audio capabilities, communication devices, and so on. Such an audio input unit 81 may also be arranged to be connected to an internet and to receive such audio and/or control signals. The audio output unit 83 may similarly be fabricated of any conventional articles which are capable of converting electrical and/or optical signals into audible acoustic waves such as, e.g., speakers. Such an audio output unit 83 may generate sound in a mono or stereo modes. In the embodiment of FIG. 1A, both of the audio input and output units 81, 83 are incorporated between the distal end of the second unit 22 and input unit 71. Such units 81, 83, however, may be disposed in other locations of the body member 20 as long as the audio input unit 81 may readily acquire various audio and/or control signals and the audio output unit 83 may readily play such sound. Accordingly, such audio input and/or output units 81, 83 may be disposed away from the input unit 71 or the grip area of the second unit 22 when feasible.

Although not included in the figure, the control member may further include at least one storage unit which may receive and store various audio and/or control signals therein. The storage unit may be fabricated of any conventional data storage articles such as, e.g., magnetic tapes, magnetic disks, optical disks, microchips, and other data storage devices for temporarily or permanently storing analog and/or digital data therein. Depending upon types of such articles, the control member may also have suitable drivers to operate such a storage unit, where examples of such drivers may include, but not be limited to, magnetic or optical tape drivers, magnetic or optical disk drivers, and so on. The storage unit may be disposed in various parts of the body member 20 and, therefore, may be disposed inside or on the first and/or second units 21, 22.

In operation, the first and second units 21, 22 are fixedly or movably coupled to each other and form the body member 20. The system 10 is then connected to an electric power outlet with a power supply cable (not included in the figure) or provided with a battery disposed inside the first or second unit 21, 22. Using the audio input unit 81, the user may record desirable sound in the storage unit or may import the audio signals thereto from the external audio or communication devices, from external data storage articles, through an internet, and the like. The user may then insert the head 21H of the first unit 21 of the body member 20 into such a pelvic cavity. When the user contacts the input unit 71 and/or presses the input unit 71, the input unit 71 begins to generate the sensing signals based upon the dynamic pattern of the user input and to deliver the sensing signals to the control member which manipulates a stimulator unit (not shown in the figure). Depending on dynamic characteristics thereof, the stimulator unit may effect one or multiple movements of a single part or multiple parts of the body member 20 and/or one or multiple movements of at least a substantial part of the body member 20, thereby providing the same or different stimuli to one or more portions of the pelvic structure. Based on such user input and/or movements of such parts, the control member manipulates the audio output unit 83 to generate the sound by playing one or more of the stored audio signals or by relaying such audio signals from the above external devices or internet.

In another exemplary embodiment of this aspect of the invention and as described in FIG. 1B, an exemplary pelvic relaxing system 10 includes a body member 20 with a first unit 21 and a second unit 22, an actuator member disposed inside the body member 20 (thus not shown in this figure), and a control member with an input unit 71, an audio input unit 81, multiple audio output units 83, and other units incorporated in the body member 20 (thus not shown in this figure). The first unit 21 is similar to that of FIG. 1A, and defines a head 21H, a trunk 21T, and a base 21B. An entire part or at least a part of the base 21B, however, is arranged to form a recess with a diameter and/or thickness which may be less than that of a lumen (denoted by dotted lines) defined in a proximal part of the second unit 22. Accordingly, the body member 20 may be arranged to dispose the recessed part of the base 21B into and out of the lumen of the second unit 22, thereby effecting translational movements of the first unit of the body member 20. The second unit 22 of FIG. 1B is elongated and forms a shape and/or size to form the grip for the user, whether the user grabs the second unit 22 with his or her thumb pointing proximally, distally, and/or laterally.

The input unit 71 movably couples with a distal end of the second unit 22 and is also oriented so that an user may apply an user input thereto through at least an area of the input unit 71 vertically, horizontally, an angle or angularly. The input unit 71 is also arranged to move between more than two states, thereby operating similar to conventional joysticks. Such an input unit 71 is arranged to monitor various dynamic patterns of the user input such as, e.g., its movement effected by the user and to generate sensing signals based upon such dynamic patterns. The input unit 71 is also shaped and/or sized such that the user may manipulate the input unit 71 while grabbing or holding the second unit 22 whether the user grabs such a unit 22 with his or her thumb pointing proximally, distally, and/or laterally. More specifically, such an input unit 71 generates the sensing signals of which amplitudes, durations or other temporal and/or spatial patterns may be determined by various dynamic patterns of the user input for grabbing and holding the second unit 22. Accordingly, the user may control various features of pelvic relaxing operation while grabbing and holding such a second unit 22 during use and without having to change the grip of the second unit 22, without having to move the hand to reach the input unit 71 while holding or grabbing the second unit 22, and the like.

The control member also includes the audio input unit 81 and a pair of audio output units 83A, 83B. The audio input unit 81 is similar or identical to that of FIG. 1A and acquires audio and/or control signals from various sources, while the first audio output unit 83A disposed in the second unit 22 is similar to that of FIG. 1A generates sound based on such acquired audio and/or control signals. The second audio output unit 83B is similarly arranged to generate sound from the acquired audio and/or control signals. However, the second audio output unit 83B is disposed in the first unit 21 and, more specifically, in the distal end of the first unit 21 or a location which tends to contact the clitoris of the user when engaged with the pelvic structure. The main function of such an audio output unit 83B is to generate and deliver acoustic waves to a specific portion of the pelvic structure, thereby providing different stimuli (i.e., acoustic stimuli) to such a portion. The audio output unit 83B of such a type may also be disposed in other locations of the body member 20 as long as such an audio output unit 83B may readily deliver the acoustic waves to various portions of the pelvic structure. Accordingly, such an audio output unit 83B may be disposed in the head 21H or trunk 21T of the first unit 21 and deliver the acoustic waves to other portions of the pelvic structure such as the G-spot or other portions on the pelvic wall. The control member may also include at least one storage unit which may be similar or identical to that of FIG. 1A.

In operation, the first and second units 21, 22 are fixedly or movably coupled with each other to form the body member 20, and the system 10 is connected to an electric power outlet with a power supply cable (not included in the figure) or provided with a battery. Using the audio input unit 81, the user may record desirable sound in the storage unit or may import the audio signals thereto from the external audio or communication devices, from external data storage articles, through an internet, and the like. The user then inserts the head 21H of the first unit 21 into the pelvic cavity. When the user pivots, swivels or otherwise manipulates the input unit 71 and moves the input unit between different states, the input unit 71 generates the sensing signals based upon one or more dynamic patterns of the user input and delivers the sensing signals to the control member which manipulates an actuator member (not included in the figure). Based on dynamic characteristics thereof, the actuator member may effect one or multiple movements of a single part or multiple parts of the body member 20 and/or one or multiple movements of at least a substantial part of the body member 20, thereby providing the same or different stimuli to one or more portions of the pelvic structure. Based upon such user input and/or movements of such parts, the control member controls the first audio output unit 83A and plays the sound or relays such audio signals from the above external devices or internet. In addition, such a control member manipulates the second audio output unit 83B and delivers the acoustic waves to a preset portion of the pelvic structure, thereby providing acoustic stimuli thereto. In the embodiment of FIG. 1B, the actuator member may be disposed inside the first and/or second units 21, 22 of the body member 20 and generate translation movement of the first unit 21 into and out of the second unit 22. Because the input unit 71 is disposed and oriented as described above, the user may effect different movements of the same or different parts in order to deliver the same or different stimuli to the same or different portions of such a structure by simply supplying different user inputs through an at least substantial identical area of the input unit 71, without moving his or her hand and/or changing the grip for controlling the input unit 71 and/or providing different user inputs thereto, without manually moving the body member 20 in the cavity for contacting the same part of the body member 20 with different portions of the pelvic structure, and so on. Other configurational and/or operational characteristics of the system of FIG. 1B are similar or identical to those of FIG. 1A.

In another exemplary embodiment of this aspect of the invention and as described in FIG. 1C, an exemplary pelvic relaxing system 10 includes a body member 20 with a first unit 21 and a second unit 22, an actuator member disposed inside the body member 20 (thus not shown in this figure), and a control member with an input unit 71, an audio input unit 81, an audio output unit 83, a driver unit 84, and various other units incorporated inside the body member 20 (thus not shown in this figure). The first unit 21 is similar to that of FIG. 1A, but includes at least one bulge 23 which is formed in the base 21B and strategically shaped and/or sized to contact a clitoris of the pelvic structure. An entire part or at least a part of the bulge 23 is thereby arranged to effect vibration and/or other movements so as to deliver stimuli to the clitoris. Similar to that of FIG. 1A, the second unit 22 of FIG. 1C is elongated and defines a shape and/or size to form the grip for the user, whether the user grabs the second unit 22 with his or her thumb pointing proximally, distally, and/or laterally.

The input unit 71 is movably disposed on a side of the second unit 22 and oriented such that an user may apply an user input thereto through at least an area of the unit 71 vertically, horizontally, an angle, and/or angularly. Such an input unit 71 is also arranged to operate between more than two states similar to conventional direction-sensitive switches, to monitor various dynamic patterns of the user input (e.g., its movement effected by the user), and to generate sensing signals based upon such dynamic patterns. The input unit 71 is also shaped and/or sized so that the user may manipulate the input unit 71 while grabbing or holding such a second unit 22, whether the user grabs such a unit 22 with his or her thumb pointing proximally, distally, and/or laterally. Thus, the input unit 71 generates such sensing signals of which amplitudes, durations or other temporal and/or spatial patterns may be determined by various dynamic patterns of the user input for grabbing and holding the second unit 22. Thus, the user may control various features of pelvic relaxing operation while grabbing or holding the second unit 22 during use and without having to change the grip of the second unit 22, without having to move his or her hand to reach the input unit 71 while holding and grabbing the second unit 22, and the like. When desirable, the input unit 71 may define multiple areas such as a center area 72C and a peripheral area 72P each of which may generate different sensing signals in response to the same or different user inputs, thereby allowing the user to control more features of pelvic relaxing operation. It is appreciated, however, that such multiple areas 72C, 72P may be arranged so that manipulating one of such areas 72C, 72P may result in manipulating the other thereof 72C, 72P, while keeping the same operation of the other area or changing the operation of the other area.

The control member also includes the audio input unit 81, audio output units 83, and driver unit 84. The audio input unit 81 is similar or identical to that of FIG. 1A and acquires audio and/or control signals from various sources, while the audio output unit 83 is similar to that of FIG. 1A and generates sound based on such acquired audio and/or control signals. The driver unit 84 is disposed between the bulge 23 and main stem of the second unit 22 and arranged to generate movement of the bulge 23 in response to the sound or acquired audio and/or control signals. More specifically, the driver unit 84 is arranged to effect the movement of the bulge which may be different from another movement of the bulge effected by the actuator member. Thus, the main function of such a driver unit 84 is to generate the auxiliary movement of the bulge 23 and to deliver different stimuli to a specific portion of the pelvic structure. Such a driver unit 84 may be disposed in other locations of the body member 20 as long as the driver unit 84 may readily effect the auxiliary movement of a specific part of the body member 20 and deliver such auxiliary stimuli to various portions of the pelvic structure. It is preferred, however, that the driver unit 84 effect such auxiliary movement mainly based upon the sound generated by the control member and/or the aforementioned audio and/or control signals acquired by the audio input unit 81. The control member may also include at least one storage unit which may be similar or identical to that of FIG. 1A.

In operation, the first and second units 21, 22 are fixedly or movably coupled with each other to form the body member 20, and the system 10 is connected to an electric power outlet with a power supply cable (not included in the figure) or provided with a battery. Using the audio input unit 81, the user may record desirable sound in the storage unit or may import the audio signals thereto from the external audio or communication devices, from external data storage articles, through an internet, and the like. The user then inserts the head 21H of the first unit 21 into the pelvic cavity. When the user pivots, swivels or otherwise manipulates the input unit 71 and moves the input unit between different states, the input unit 71 generates the sensing signals based on one or more dynamic patterns of the user input and delivers the sensing signals to the control member which then manipulates an actuator member (not included in the figure). When the input unit 71 includes multiple areas 72C, 72P, the user may supply different user inputs onto different areas 72C, 72P while keeping or changing operations determined by such areas 72C, 72P. Based on dynamic characteristics thereof, the actuator member effects one or multiple movements of a single part or multiple parts of the body member 20 and/or one or multiple movements of at least a substantial or entire part of the body member 20, thereby providing the same or different stimuli to one or more portions of the pelvic structure. In the embodiment of FIG. 1C, the actuator member is disposed inside the body member 20, thereby effecting vibration or other movements of the bulge 23, in addition to various optional movements of at least a part of such a first unit 21. Based on such user input and/or movements of such parts, the control member controls the audio output unit 83 and generates such sound or relays such audio signals from the above external devices or internet. In addition, such a control member manipulates the driver unit 84 and delivers the auxiliary stimuli onto a preset portion of the pelvic structure in response to the sound or audio and/or control signals. Because the input unit 71 is disposed and oriented as described above, the user may effect different movements of the same or different parts of the body member 20 and deliver the same or different stimuli to the same or different portions of such a structure by simply supplying different user inputs through an at least substantial identical area of the input unit 71, without moving his or her hand and/or changing the grip for manipulating the input unit 71 and/or providing different user inputs thereto, without manually moving the body member 20 in the pelvic cavity for contacting the same part of the body member 20 with different portions of such a structure, and so on. Further configurational and/or operational characteristics of the system shown in FIG. 1C may be similar or identical to those of FIGS. 1A and 1B.

In another exemplary embodiment of this aspect of the invention and as described in FIG. 1D, an exemplary pelvic relaxing system 10 includes a body member 20 with a first unit 21 and a second unit 22, an actuator member disposed inside the body member 20 (thus not shown in this figure), and a control member with an input unit 71, a first audio input unit 81A, a second audio input unit 81B, an audio output unit 83, a driver unit 84, and various other units incorporated inside the body member 20 (thus not shown in this figure). The first unit 21 is similar to that of FIG. 1C, but has multiple bulges 23 which are defined about different parts of the body member 20 in various intervals and strategically shaped and/or sized to contact the G spot along the pelvic structure. More specifically, an entire part or at least a part of such bulges 23 may be arranged to effect vibration and/or other movements so as to deliver stimuli to the G spot. Similar to that of FIG. 1A, the second unit 22 is elongated and defines a shape and/or size to define the grip for the user, whether the user grabs the second unit 22 with his or her thumb pointing proximally, distally, and/or laterally.

The input unit 71 includes a receiving area 72 and multiple tracks 72T and is disposed on a side of the second unit 22. More specifically, the input unit 71 has two parallel elongated tracks 72T which are connected to each other by a transverse track 72T. The receiving area 72 is movably disposed in the tracks 72T and moves along the tracks 72T in response to an user input applied to such an area 72 vertically, horizontally, an angle, and/or angularly. The input unit 71 is also arranged to operate between more than two states and to monitor other dynamic patterns of the user input such as, e.g., force of the user input, its velocity, its acceleration, and other temporal and spatial patterns thereof. The input unit 71 is also shaped and/or sized such that the user may manipulate the receiving area 72 while grabbing or holding such a second unit 22 whether the user grabs such a unit 22 with his or her thumb pointing proximally, distally, and/or laterally. Accordingly, the user may control various features of pelvic relaxing operation while grabbing or holding the second unit 22 during use, without having to change the grip of the second unit 22, without having to move the hand to manipulate the input unit 71 while holding and grabbing the second unit 22, and the like. Similar to that of FIG. 1C, the input unit 71 may define multiple receiving areas such as, e.g., a center area and a peripheral area each capable of generating different sensing signals in response to the same or different user inputs, thereby allowing the user to control more features of pelvic relaxing operation.

The control member also includes a pair of audio input units 81A, 81B and a single audio output unit 83. The first audio input unit 81A disposed in the second unit 22 is generally similar or identical to that of FIG. 1A and acquires audio and/or control signals from various sources, while the audio output unit 83 is similar to that of FIG. 1A and generates sound based on such acquired audio and/or control signals. The second audio input unit 81B is similarly arranged to acquire the audio signal. However, the second audio input unit 81B is disposed in the first unit 21 and, more specifically, in the head 21H or trunk 21T thereof or another location which tends to contact other portions of the pelvic wall when engaged with the pelvic structure. The main function of such an audio input unit 81B is to acquire the sound generated between the pelvic wall and first unit 21 of the body member 20, thereby providing audio signals representing physiological states inside the pelvic cavity. Such an audio input unit 81B may also be disposed in other locations of the body member 20 as far as the audio input unit 81B may readily acquire the sound from various portions of the pelvic structure. Thus, such an audio input unit 81B may be disposed in the base 21B of the first unit 21 and acquire the sound from other portions of the pelvic wall such as the G-spot. One or multiple driver units 84 are disposed between the bulge 23 and main stem of the second unit 22 and arranged to generate movement of the bulge 23 in response to such sound or acquired audio and/or control signals. In general, such driver units 84 are similar or identical to that of FIG. 3C. The control member may also include at least one storage unit which may be similar or identical to that of FIG. 1A.

In operation, the first and second units 21, 22 are fixedly or movably coupled with each other to form the body member 20, and the system 10 is connected to an electric power outlet with a power supply cable (not shown in the figure) or provided with a battery. Using the first audio input unit 81A, the user may record desirable sound in the storage unit or may import the audio signals thereto from the external audio or communication devices, from external data storage articles, through an internet, and the like. The user then inserts the head 21H of the first unit 21 into the pelvic cavity. As the user controls the receiving area 72 along a first track 72T, the input unit 71 generates the sensing signals based on a position of the receiving area 72 on the track or another dynamic pattern of the user input and delivers such signals to the control member which manipulates an actuator member (not included in the figure). When a specific dynamic feature of such movement of the body member 20 attains a desirable state, the user may set such a setting by, e.g., pressing the receiving area 72 in a current position, activating a separate set button (not shown in the figure), and the like. The user may move the receiving area 72 to a second track 72T across the transverse track, move the area 72 along the second track 72, and set a new position when another dynamic feature of the movement of the body member 20 attains a desirable state. Based on dynamic characteristics thereof, the actuator member may effect one or multiple movements of a single part or multiple parts of the body member 20 and/or one or multiple movements of at least a substantial part of the body member 20, thereby providing the same or different stimuli to one or more portions of such a pelvic structure. The user may also obtain the sound generated by operation of the system in the pelvic cavity through the second audio input unit 81B, where such sound may be delivered to the user or may be used to control other members or units of such a system 10 as a feedback variable. In the embodiment of FIG. 1D, the actuator member may effect vibration or other movements of at least a part of the first unit 21. When the input unit 71 is disposed and also oriented described above, the user may effect different movements of the same or different parts of such a body member 20 and then deliver the same or different stimuli to the same or different portions of the pelvic structure by supplying different user inputs through a single receiving area of the input unit 72, without moving the hand and/or changing the grip for controlling the input unit 71 and/or providing different user inputs thereto, without having to manually move the body member 20 in the pelvic cavity for contacting the same part of such a body member 20 with different portions of the pelvic structure, and the like. Other configurational and/or operational characteristics of such a system of FIG. 1D may be similar or identical to those of FIGS. 1A to 1C.

Configurational and/or operational variations and/or modifications of the above embodiments of the exemplary systems and various members thereof described in FIGS. 1A through 1D also fall within the scope of this invention.

The body member and its units may be fabricated similar or identical to shapes and/or sizes of any conventional pelvic relaxing devices. Accordingly, the first unit may have various shapes, sizes, and curvatures which may be constant along an entire portion of a longitudinal axis of the first unit or may vary therealong. Further details of such shapes, sizes, curvature, and other configurations are provided in the above prior art patents and/or publications incorporated herein by reference. Similarly, the second unit may have various shapes, sizes, and curvatures which may also be constant along the entire portion of the axis or may also vary therealong. It is appreciated, however, that handles of the conventional pelvic relaxing devices are generally longer in order to accommodate a space for the grip and another space for incorporating various switches. In contrary, the pelvic relaxing system of this invention incorporates the input unit disposed in the same area as the grip therefor and, therefore, the second unit of such a system may typically be shorter than its conventional counterparts. Such a body member and its first and second units may also be made of and/or include materials as employed in any conventional pelvic relaxing devices. Accordingly, the first unit may be made of and/or include flexible or rigid materials, soft or hard materials, composite materials, and the like. When desirable, the first unit may be arranged to exhibit different mechanical properties in different parts thereof. The first unit may further form at least one internal lumen in which various members and/or units of the system may be disposed. The second unit may also be made of and/or include flexible or rigid materials, soft or hard materials, as long as such an unit may form the grip for the user.

Although the pelvic relaxing systems of FIGS. 1A to 1D exemplify combinations of various first units, second units, input units, audio input units, audio output units, and driver units, these units may be used in different combinations. For example, the audio input units of the system of FIG. 1D may be incorporated into the systems of FIGS. 1a to 1C, and their body members 20 may include such audio input units in the same or different locations. Such a system may incorporate different combinations of various body, actuator, and/or control members as will be described in greater detail below.

Further details of various members and/or units of such pelvic relaxing systems of FIGS. 1A to 1D are provided in the first of the aforementioned co-pending Applications.

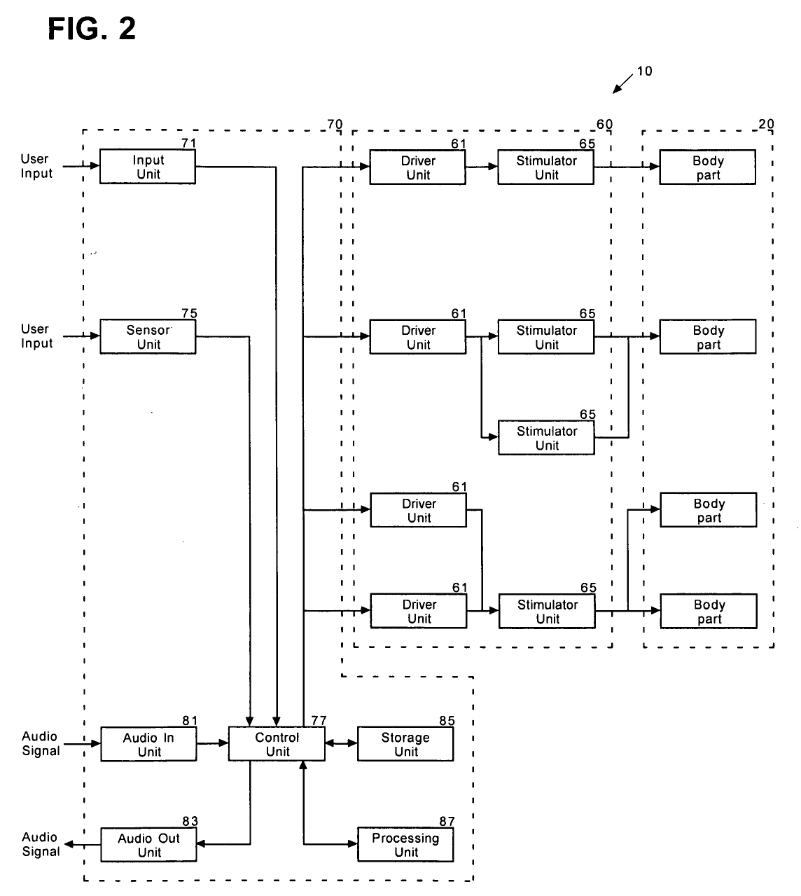

In another aspect of the present invention, an exemplary pelvic relaxing system may include at least one body member, at least one actuator member, at least one control member, and at least one power (supply) member. FIG. 2 is a schematic diagram of various functional members and units of an exemplary pelvic relaxing system according to the present invention. A typical pelvic relaxing system 10 is comprised of at least one body member 20, at least one actuator member 60, at least one control member 70, and at least one power (supply) member (not included in the figure) which may be a wire and plug assembly for receiving AC power from an electric outlet or may instead be a dry-cell battery or a rechargeable battery.

As briefly described in FIGS. 1A to 1D, the body member 20 includes at least one first unit and at least one second unit, where the first unit defines various parts capable of contacting one or more portions of the pelvic structure when engaging therewith and providing various stimuli through one or more movements thereof. The body parts with various configurations are generally similar to those of the prior art devices, whereas various novel body parts are provided in the co-pending Applications.

The actuator member 60 includes at least one driver unit 61 and at least one stimulator unit 65. The main function of the actuator member 60 is to effect one or multiple movements of one or multiple parts of the body member 20 (i.e., the body parts of the first unit thereof). To this end, the driver unit 61 receives electric power from the power member and generates driving force which is transferred to the stimulator unit 65 through at least one power transmission unit (not included in the figure). The driving unit 61 may be a conventional electric motor for generating rotational movement of its axle, an electromagnetic vibrator assembly for generating reciprocating movement of a mobile assembly. The rotational movement of the axle of the electric motor may be converted by various power conversion units to effect various driving forces. When the system is to effect multiple movements, the actuator member 60 may include multiple driver units 61 each effecting one or more of such movements either independently or in cooperation. The stimulator unit 65 is then arranged to receive the driving force, to optionally convert the driving force into actuating force, and to effect desirable movement of a preset part of the first unit of the body member 20 by such actuating force. To this end, the stimulator unit 65 mechanically, electrically, and/or magnetically couples with the preset part of the body member 20 and ensures its actuating force to effect desirable movement of the body part. The actuator member 60 may also include the power transmission unit which may transmit the driving and/or actuating forces from one to another locations of the body member 20 while maintaining or changing amplitudes and/or directions of the forces. The power transmission unit may be any conventional articles of commerce capable of transmitting the rotational and/or translational movements therealong, and examples of the power transmission unit may include, but not be limited to, assemblies of multiple axles coupled by one or more universal joints, rigid or flexible metal cables, and the like. Such an actuator member 60 may also include one or multiple driver and/or stimulator units 61, 65 which may be operatively coupled to each other in various modes. In one example, a single driver unit 61 may be operatively coupled to a single stimulator unit 65 and deliver such driving force thereto. In another example, a single driver unit 61 may be operatively coupled to two or more stimulator units 65 and deliver the same driving force to multiple stimulator units 65 one at a time or simultaneously. The actuator member 60 may also include one or more power conversion units for delivering different driving forces to different stimulator units 65. In another example, two or more driver units 61 may be operatively coupled to a single stimulator unit 65 and deliver different driving forces thereto one at a time or simultaneously. When desirable, at least two of the driver units 61 may be arranged to drive the stimulator unit 65 simultaneously so as to effect compound actuating force.

Similarly, the actuator member 60 may be coupled to the body member 20 in various modes. In one example, a single stimulator unit 65 may operatively couple with a single body part and deliver the driving and/or actuating forces thereto. In another example, a single stimulator unit 65 may operatively couple with two or more body parts and deliver the same driving or actuating forces to multiple body parts one at a time or simultaneously. In another example, multiple stimulator units 65 may operatively couple with a single body part and deliver different driving or actuating forces to such a body part one at a time or simultaneously. When desirable, at least two of the stimulator units 65 may be arranged to move the body part simultaneously so as to effect compound movement of such a part. The actuator member 60 may manipulate various movements of the single part or multiple parts of the body member 20 by its single or multiple stimulator units 65 by various actuating forces. For example, the actuator member 60 may be arranged to generate various types of movements of the single or multiple units (or their parts) of the body member 20. In one example, the actuator member 60 may generate vibration of at least a substantial part of the first unit of the body member 20, vibration of at least one but not all of the head, trunk, and base of the first unit, vibration of one or multiple bulges designated to stimulate the clitoris, G-spot, pelvic wall, and so on. In another example, the actuator member 60 may generate translation of at least a substantial part of such a first unit, translation of at least one but not all of the head, trunk, and base of the first unit, translation of such bulges, and the like, where such translation may be effected through and/or around the pelvic opening for stimulating such clitoris, G-spot, pelvic wall, and the like, and where such translation may be parallel to, perpendicular to or at an angle with respect to the pelvic opening and/or longitudinal axis of the first unit. In another example, the actuator member 60 may generate rotation or pivoting of at least a substantial part of the first unit, rotation or pivoting of at least one but not all of the head, trunk, and base of such a first unit, rotation or pivoting of such bulges, and the like. It is appreciated that such rotation or pivoting may be effected about the longitudinal axis of the first unit or, alternatively, may be effected about a center of rotation or an axis of rotation which may be formed in the first or second units of the body member 20. Therefore, such rotation or pivoting may be effected to the clitoris, G-spot, and pelvic wall when desirable. In addition and depending on the location of such a center, the rotation or pivoting may be effected as swivelling of an entire or only a part of the first unit. In another example, the actuator member 60 may generate tapping or thumping of at least a substantial part of the first unit, tapping or thumping of at least one but not all of the head, trunk, and base of the first unit, tapping or thumping of the bulges, and the like. Therefore, the actuator unit 60 may provide such tapping or thumping stimuli to the clitoris, G-spot, and pelvic wall. In another example, such an actuator member 60 may generate deformation of at least a substantial part of the first unit, deformation of at least one but not all of such head, trunk, and base of the first unit, deformation of one or more of the bulges, and so on. Other configurational details of the body and actuator members 20, 60 for effecting the above movements are provided in the first of the co-pending Applications.

The control member 70 may include at least one control unit 77 and at least one of at least one input unit 71 and sensor unit 75. The main function of the control member 70 is to generate signals for manipulating various units of the actuator member 60. To this end, the input and/or sensor units 71, 75 may be arranged to receive various user inputs and to generate sensing signals in response thereto, while the control unit 77 may be arranged to generate control signals according to the sensing signals such that the actuator member 60 may generate the driving and/or actuating forces in response to the user inputs. Accordingly, the stimulator units 65 of the actuator member 60 may actuate the body part in order to deliver desired stimuli to a single or multiple portions of the pelvic structure. As have been disclosed in the first of the co-pending Applications, the input and sensor units 71, 75 basically refer to similar articles capable of receiving various user inputs and generating various sensing signals in response thereto. Within the scope of this invention, those articles incorporated into the first unit of the body member are to be referred to as the sensor units 75, whereas those incorporated into the second unit of the body member will be referred to as the input units 71, unless otherwise specified. Other configurational details of the input and sensor units 71, 75 of the control member 60 as well as further control operations of the control member 60 have been disclosed in the first of the co-pending Applications and will be omitted for ease of illustration.

As described in conjunction with FIGS. 1A to 1D, the control member 70 also includes at least one audio input unit 81, audio output unit 83, storage unit 85, signal processing unit 87, and other units such as the driver unit. The audio input unit 81 may have any shapes and/or sizes and any number of such units 81 may be incorporated into the system 10 as long as the audio input unit 81 may obtain the audio and/or control signals from various sources of signals and/or sound as described above. When desirable, the audio input unit 81 may be operatively coupled to microphones of external devices and then import the audio and/or control signals directly therefrom through wire or wirelessly. In addition, the control member 70 may have different audio input units 81 to obtain different types of audio and/or control signals. The audio input unit 81 is typically disposed in the second unit of the body member 20, although such may be disposed in the first unit as exemplified in FIG. 1D. The audio output unit 83 may have any shapes and/or sizes and any number of such units 83 may be incorporated into the system 10 as long as the audio output unit 83 may generate sound based on the audio and/or control signals obtained from various sources of signals and/or sound as described above. When desirable, such an audio output unit 83 may be operatively coupled to speakers of external devices and then export such audio and/or control signals directly thereto through wire or wirelessly, thereby generating the sound with the external speaker. In addition, the control member 70 may have different audio output units 83 to generate different types of sounds. Moreover and as depicted in FIG. 1B, the audio output unit 83 may be arranged to provide the acoustic stimuli directly to a preset portion of the pelvic structure. The audio output unit 83 is preferably arranged to contact or touch the desired portion of the structure so as to deliver such stimuli thereto with the least loss or attenuation of the waves. In addition, such an audio output unit 83 may be arranged to directly or indirectly actuate the driver unit exemplified in FIGS. 1C and 1D as well.

The storage unit 85 may store various audio and/or control signals temporarily or permanently. Such an unit 85 may be fabricated from any conventional data storage articles such as, e.g., magnetic tapes, magnetic disks, optical disks, semiconductor chips, and other data storage devices for storing analog and/or digital data therein. Depending upon types of the articles, the control member may also include suitable drivers to operate the storage unit 85, where examples of the drivers may include, but not be limited to, magnetic tape drivers, optical tape drivers, magnetic disk drivers, optical disk drivers, circuits for recognizing and retrieving desired signals, and the like. Such signals may be classified in various modes, e.g., based upon contents of such signals, their classifications, presence or absence of action bases therein, source thereof, their voice bases, and the like. Therefore, the control member 70 may readily find and then retrieve the desired signal from such a storage unit 85. The storage unit 85 may be disposed in various locations of the system 10 and may be exposed or hidden in the first and/or second units of the body member 20. When desirable, the storage unit 85 may be provided as a replaceable cartridge so that the user may load a desired storage unit 85, change such a unit 85 full of capacity, and the like. The control member 70 may also be arranged to communicate with external storage article and to send, store, search, and/or retrieve desired signals thereto or therefrom.

It is to be understood within the scope of the present invention that any audio signal is deemed to define at least one of a content basis, a voice basis, an action basis, and a background basis. The content basis refers to any word or phrase of the user or third party which may or may not carry any meaning, and the voice basis means wave characteristics of the audio signal of the user or third party characterizing audible waves generated by human vocal cords and/or those produced by mechanical equivalents of the vocal cords. In contrary, the action basis represents characteristics of the sound accompanying actions of the user or third part and may contain neither the content basis nor the voice basis. Finally, the background basis means none of the above such as, e.g., music, melody, and the like. It is appreciated that non-instrumental music such as opera may be deemed as sound composed of audio signals with the voice basis superposed to the background basis. With these definitions, the signal processing unit 87 receives the audio signals and modifies the audio signals by changing, e.g., at least on temporal pattern thereof, their amplitudes, their frequencies, their orders, and so on. Such a processing unit 87 may also generate compound signals by combining multiple audio signals or, in the alternative, may generate synthesized signals by changing at least one basis of the audio signals, and the like.

As depicted in FIG. 2, the control unit 77 receives the first audio and/or control signals through the audio input unit 81 and sends the second audio and/or control signals to the audio output unit 83. It is appreciated that the control unit 77 may send to the audio output unit 83 the same first audio and/or control signals received thereby from the audio input unit 81 so that the first and second audio and/or control signals may become identical. In the alternative, the control unit 77 may receive the first audio and/or control signals and send different audio and/or control signals to the audio output unit 83 such that the second signals are different from the first signals. In the latter embodiment, the control unit 77 may change at least a portion of such first signals with or without using the signal processing unit 87, may retrieve different signals from the storage unit 85 based upon the first signals and then send the retrieved signals to the audio output unit 83, and the like.

In contrary to the audio input and output units 81, 83, the storage and processing units 85, 87 may send various audio and/or control signals to the control unit 77 and receive the same or different audio and/or control signals from the control unit 77 as well. That is, the storage and processing units 85, 87 may effect the control unit 77 as well as control member 70 such that such a unit 77 or member 70 may manipulate the actuator member 60 and/or body member 20 in different mechanisms to effect different movements of a single or multiple parts of the body member 20, to generate different stimuli by the same or different parts of the body member 20, to provide various stimuli to a single or multiple portions of the pelvic structure, to generate different sounds as a response to various user inputs or movements of the body parts, and the like. Various control mechanisms of such a control unit 77 and member 70 will now be described in greater detail below.

First of all, the control member 70 may obtain the audio and/or control signals through various sources. In one example and as described above, the control member 70 may retrieve pre-recorded signals from the storage unit 85. When the retrieved (or internal) signals are audio signals, the audio output unit 83 may directly play such signals and generate the sound. When such internal signals are control signals, the audio output unit 83 may generate the sound while controlling one or more bases of the sound based on the internal control signals. It is appreciated that the storage unit 85 may store therein such audio and/or control signals which may be analog or digital signals and that such an unit 85 may store the signals with or without data compression. The stored signals may be related to the user or third party. In another example, the control member 70 may be operatively coupled to external data storage devices so as to retrieve the pre-recorded audio and/or control signals therefrom. When desirable, the control member 70 may include appropriate drivers to operate the data storage devices or, alternatively, may be arranged to manipulate external drivers to operate the data storage devices. In another example, such a control member 70 may obtain the audio or control signals through various external audio devices. Such signals are typically pre-recorded signals and may also be related to the user and/or third party. In another example, the control member 70 obtains such audio and/or control signals through various external communication devices. Such signals may be pre-recorded signals or live signals and related to the user or third party. In another example, the control member 70 may receive the pre-recorded or real-time audio and/or control signals of the user or third party through an internet. The control member 70 may download such signals and then manipulate the audio output unit 83 based thereon or may directly generate the sound in response thereto in real time. In yet another example, the control member 70 may generate the compound and/or synthesized signals from multiple audio and/or control signals so as to generate the sound therefrom. Once obtaining such audio and/or control signals, the control member 70 may process the audio and/or control signals and generate the desired sound according to the procedures exemplified in the first example of this paragraph. It is to be understood that such audio and/or control signals may be analog or digital signals and may also be compressed when desirable.

Secondly, the control member 70 may turn on or activate the audio output unit 83 and generate the sound based upon activation of the system 10 and/or its various members. In one example, such a control member 70 may turn on the audio output unit 83 during the system 10 is in use which may be signified when a main switch is turned on, when the actuator member 60 or its stimulator units 65 are activated, and the like. The control member 70 may also activate the audio output unit 83 when one or more specific input and/or sensor units 71, 75 receive a specific user input or any user input through a preset area or any area thereof, when one or more dynamic patterns of such user input reaches or exceeds a preset threshold, and the like. The control member 70 may further activate the audio output unit 83 when the first unit of the body member 20 is engaged with the pelvic structure, when at least a preset length of the first part is inserted into the internal cavity, when the user generates a motion or movement of the entire system 10 to or beyond a preset acceleration, velocity, and/or displacement, and the like.

The control member 70 may also manipulate the audio output unit 83 to generate specific sound for a preset period of time using a single audio signal or multiple audio signals in a random order or in a preset order, to generate different sounds for a preset period of time in a random order or in a preset order, and in other manners as will be described in greater detail below, in response to various user inputs, audio signals, control signals, and/or other signals. In one example, the control member 70 may generate such sound in such a manner based on the user input (or its dynamic pattern) applied to the input unit 71 or sensor unit 75. Therefore, the user or third party may curtail their input in such a way to effect desired sound in the desired mode. In another example, the control member 70 may generate such sound in such a manner based upon one or more dynamic features of movement of a single part or multiple parts of the body member 20. Therefore, the audio output unit 83 may generate different sound depending upon, e.g., a speed of vibration of such body parts, a displacement distance of the first unit, and the like. In another example, the control member 70 may generate such sound in such a manner based on the state of the system 10, whether such a system 10 is turned on or off, whether the actuator and/or control members 60, 70 are turned on or off, and the like. In another example and when the system 10 include multiple actuator members 60 and/or stimulator units 65 and/or when the body member 20 defines multiple parts, the control member 70 may also generate such sound in such a way based upon which actuator member 60, stimulator unit 65, and/or part may be selected by the user input for effecting a single movement or multiple movements. When the actuator members 60 or their stimulator units 65 may generate multiple different movements, the control member 70 may further generate such sound in such a manner depending on which movement may be selected by the user input. In another example, the control member 70 may generate such sound in such a manner based upon states of engagement of the system 10 with the pelvic structure. Therefore, the system 10 may activate the audio output unit 83 and generate the sound when a preset part of the first unit is inserted into the internal cavity, a preset part of the first unit engages with the clitoris, and the like. In another example, the control member 70 may generate such sound in such a manner based upon a type or an extent of movement of the system as a whole so that the audio output unit 83 may be activated so as to generate the same sound in different patterns, to generate different sounds in response to various dynamic features of the movement of the single or multiple parts of the body member 20, and the like. In another example, the control member 70 may activate the audio output unit 83 and manipulate such an unit 83 to generate such sound in such a manner in response to the user input carried in the voice of the user or third party. As far as the control member 70 may recognize such an audible user input, such a member 70 may also perform any of the above and following control mechanisms through the audible audible user inputs. In another example, the control member 70 may also manipulate the audio output unit 83 based on the external audio and/or control signals which may be pre-recorded or real-time and which may delivered to the control member 70 through the external audio or communication devices, internet, and the like.

Such a control member 70 may also generate the compound and/or synthesized audio and/or control signals and generate the sound therefrom in such a manner. Accordingly, the control member 70 may play the sound which defines only one of such content, voice, action, and background bases, another sound defining two or more of the content, voice, action, and background bases, and the like. The control member 70 may further be arranged to change only one basis of such signals and to play such modified signals to generate such sound, to impose at least one basis of first signals to second signals as described above, and so on. The control member 70 may generate the synthesized sound by altering only one (or multiple) basis of the signals but not all bases thereof. The control member 70 may perform such synthesis through the signal processing unit 87.

The control member 70 preferably synchronizes various sounds generated by the audio output unit 83 with numerous events such as, e.g., various operational states of the system 10, its members, and/or its units, various dynamic features of the movements of the single or multiple parts of the body member 20, various dynamic patterns of the user input, and so on. For example, the sound generated by the audio output unit 83 may be temporarily synchronized with such events so that such an unit 83 generates the sound simultaneously with at least one of such events, immediately after at least one of such events, in a preset interval after at least one of such events, and the like. Similarly, such an unit 83 may be arranged to generate such sound as long as at least one of such events persists, to stop generating such sound simultaneously with cessation of at least one of such events, immediate after cessation of at least one of such events, in a preset interval after cessation of at least one of such events, and the like. Alternatively, the audio output unit 83 may stop generating such sound only after it receives another user input. In addition, the event actuating the audio output unit 83 may not be the same event terminating such an unit 83. Therefore, the audio output unit 83 may start to generate the sound with one event and then stop to generate such sound with another event. In another example, the sound generated by the audio output unit 83 may be spatially synchronized with such events so that the audio output unit 83 may generate the sound when the user input is applied to the input and/or sensor units in a specific spatial pattern, when such events may occur in a specific spatial mode, and the like.

Moreover, the control member 70 may generate such sound based upon various audio and/or control signals obtained from various sources. In one example, the audio output unit 83 may generate the sound based upon internal audio and/or control signals stored in the storage unit 85 and retrieved by the control member 70. In another example, the audio output unit 83 may generate the sound based upon the audio and/or control signals supplied thereto by the user in real time, either in the form of the user input or as a verbal (or voice) command. In another example, such an unit 83 may generate the sound based upon various audio and/or control signals obtained from the external communication or audio devices or from the internet, whether such signals may be pre-recorded ones or real-time ones. In another example, the audio input unit 83 may generate the sound based upon the compound and/or synthesized signals obtained from processing multiple audio and/or control signals.

The control member 70 may also manipulate the audio output unit 83 to provide various stimuli to various portions of the pelvic structure. In one example, the audio output unit 83 may be arranged to emit the acoustic waves of the sound directly onto the portion of the pelvic structure so that such a portion may receive the acoustic stimuli therefrom. In another example, the audio output unit 83 may include at least one vibrating or otherwise moving plate, emit the acoustic waves of such sound onto the plate, and generate vibration or movement of such a plate. By disposing the audio output unit 83 and its plate close to the portion of the pelvic structure, another stimuli may be provided to the portion of the pelvic structure. It is appreciated that characteristics of such stimuli may depend upon detailed mechanical configuration of such a plate. In another example, the control unit 70 may include at least one auxiliary driver unit capable of generating an auxiliary movement of at least one part of the body member 20. Such auxiliary movement may be delivered to the desired portion of the pelvic structure independently or may be superposed onto the movement of the part effected by the actuator member 60 or its stimulator unit 65.

Furthermore, the control member 70 may generate various sounds for a preset period of time by manipulating its various units. In one example, the control member 70 may control the audio output unit 83 to repeat a preset audio signal or a set of audio signals during the period. In another example, the control member 70 may control the audio output unit 83 to play and to repeat, as necessary, one or more audio signals randomly, in a preset order, or in a variable order as determined by the user input or other events as described above. In another example, the control member 70 may control the audio output unit 83 to play and then to repeat, as necessary, different audio signals based on the external audio and/or control signals which may be pre-recorded or real-time, which may be related to the user or third party, and which may be provided through the external audio and/or communication devices or from the internet. In another example, such a control member 70 may control the audio output unit 83 to play different audio signals during the period depending on the user input supplied to the input unit 71 and/or sensor unit 75, based on the dynamic patterns of the user input, depending on the dynamic features of the movements of the body part, based on the movement of the entire system effected by the user or third party manually, based on various operational states of the system 10 or its members as described above, and so on. The control member 70 may also categorize multiple audio or control signals based upon their content, voice, action, and/or background bases and play such signals from a specific category or group during the period, play such signals from different groups depending on various events as mentioned above, and the like.

In addition, the control member 70 may receive such audio and/or control signals from various sources as described herein and then control various operations of the system 10 according thereto. For example, the control member 70 may control the dynamic features of the movements of the single part or multiple parts, number of the body parts recruited for the movements, and the like. The control member 70 may further control the temporal and/or spatial patterns of the sound based on such audio and/or control signals.

The pelvic relaxing system of the present invention may further be construct to be waterproof. For example, various input units of the body member may be covered by a waterproof layer or may be disposed inside the second unit of the body member. In addition, the system may be arranged to run by a rechargeable battery which may be recharged by electromagnetic induction from outside.

Unless otherwise specified, various features of one embodiment of one aspect of the present invention may apply interchangeably to other embodiments of the same aspect of this invention and/or embodiments of one or more of other aspects of this invention. Therefore, various audio input and/or output units of one pelvic relaxing of FIGS. 1A to 1D may be used interchangeably. In addition, such a system may include one or multiple audio input and/or output units of the same or different types.

Unless otherwise specified, various features of one embodiment of one aspect of the present invention may further apply interchangeably to other embodiments of other aspects of other inventions disclosed in the co-pending Applications. Therefore, the audio input and/or output units of the present invention may be used in any pelvic relaxing systems having various clitoris and/or G-spot stimulators, those incorporated with interactive capabilities, those synchronized with internal and external signals, those including electric stimulators, those incorporating adjustable body members, those with retention mechanisms, those with feedback mechanisms, those with reciprocating body members, and the like.

The pelvic relaxing systems of this invention offer various advantages over their conventional counterparts. First of all, such a system allows the user to manually or automatically generate sound while using the system. Accordingly, the user may play the desired sound while using such a system and providing various stimuli to her pelvic structure, thereby obviating the user from imaging a voice of or a word from a desired partner. Secondly, the system may play such sound in synchronization with various operational characteristics of the system. Therefore, such sound may increase or decrease in its amplitudes, vary its speed and/or tone, and/or use different words in response to the user input, movements of the entire system, physical or mental states of the user, and the like. In addition, such a system may receive or generate such sound and then manipulate various operational characteristics of the system according thereto. For example, various dynamic features of the movements of one or multiple body parts may be manipulated in response to such sound, such body parts may also engage with different portions of the pelvic structure based on such sound, and so on. Moreover, the system may generate and play the synthesized audio signals using different audio signals, thereby allowing the user to play such sound carrying the voice of a desired partner, carrying the desired content, and so on. The system may generate the sound while fulfilling various advantages of other pelvic relaxing systems disclosed in the co-pending Applications.

The above systems, methods, and/or processes of the present invention may be applied to or utilized for various purposes. As described above, such systems, methods, and/or processes may be used to play the sound while providing various stimuli to various portions of such a pelvic structure including the clitoris, G-spot, and other portions on the pelvic wall. In addition, the systems, methods, and processes of the present invention may be applied to other pelvic relaxing systems such as, e.g., systems with various clitoris and/or G-spot stimulators, systems with interactive capabilities, systems synchronized with internal and external signals, systems including electric stimulators, systems with adjustable body members, systems with retention mechanisms, systems with feedback mechanisms, systems having reciprocating body members, and the like.

The above audio relaxing system may also include at least one light unit which may be capable of providing light during use. Such a light unit may operate independent of other members and may be turned on and off by the input unit. In the alternative, the light unit may operatively couple with and/or be synchronized with the audio output unit or other units of the system such that the light unit may be turned on and off based on the user input, operations of various members and/or units of the system, internal and/or external audio and/or control signals, and the like. Similar to the audio output unit, the control member may be arranged to change an intensity of light emitted by the light unit in response to various events described above, movements of the body parts or the entire system, and so on.

Such a system may further be arranged to generate video signals and/or to be manipulated by internal and/or external video signals. The system may include a video input unit and a video output unit and display a desired image on the video output unit. Alternatively, the system may be operatively coupled to external video and/or communication devices and generate the desired image on external video output units of such devices. Various control mechanisms for the audio input and output units of the systems of this invention may then be employed such video relaxing systems as well.

It is appreciated that the Disclosure Documents which have been referred to in the section of “Cross-Reference” and bear the Ser. Nos. 611,331 and 611,023 have been referred to herein as the “co-pending applications.”

It is to be understood that, while various aspects and embodiments of the present invention have been described in conjunction with the detailed description thereof, the foregoing description is intended to illustrate and not to limit the scope of the invention, which is defined by the scope of the appended claims. Other embodiments, aspects, advantages, and modifications are within the scope of the following claims.

What is claimed is:

1. A pelvic relaxing system for providing tactile stimuli onto at least a portion of a pelvic structure while providing sound, wherein said pelvic structure includes an entry and a wall, wherein said entry is configured to define an orifice therethrough and wherein said wall is configured to include muscles and to define an internal cavity which extends inwardly from said entry and is bound by said muscles, said system comprising:
at least one body member which is configured to have a first part for contacting said portion of said pelvic structure when engaged therewith and a second part for providing a grip to an user;
at least one actuator member which is configured to effect at least one movement of said first part for providing said stimuli onto said portion of said structure through said movement; and
at least one control member which is configured to include at least one of a sensor unit and at least one input, to receive an user input defining at least one dynamic pattern, to effect said movement of said first part in response to said dynamic pattern of said user input, to include therein at least one audio output unit capable of generating said sound, and to generate said sound when said user input is supplied to said at least one of said input and sensor unit.

2. The system of claim 1, wherein said audio output unit is configured to generate said sound as said actuator member starts to provide said stimuli in response to said user input.

3. The system of claim 1, wherein said audio output unit is configured to generate said sound as at least a section of said first part is inserted into said cavity through said orifice.

4. The system of claim 1, wherein said audio output unit is configured to generate said sound as said sensor unit detects movement of at least a substantial portion of said system.

5. The system of claim 1, wherein said system further includes at least one main switch capable of moving between on- and off-state and initiating and capable of stopping operation of at least one of said actuator and control members, respectively, and wherein said audio output unit is configured to generate said sound as said main switch is moved in said on-state.

6. The system of claim 1, wherein said control member is configured to have at least one storage unit for storing therein at least one pre-recorded audio signal of at least one of said user and a third party and to control said audio output unit to generate said sound by playing said audio signal.

7. The system of claim 1, wherein said control member is configured to receive from an external audio device at least one pre-recorded audio signal of at least one of said user and a third party and to control said audio output unit to generate said sound by playing said audio signal.

8. The system of claim 1, wherein said control member is configured to receive from an external communication device at least one of a pre-recorded audio signal and real-time audio signal of a third party and to control said audio output unit to generate said sound by playing said audio signal.

9. The system of claim 1, wherein said control member is configured to receive at least one of a pre-recorded audio signal and a real-time audio signal of a third party from an internet and to control said audio output unit to generate said sound by playing said audio signal.

10. The system of claim 1, wherein said control member is configured to receive a plurality of at least one of pre-recorded audio signals and real-time audio signals of at least one of said user and a third party, to generate a compound signal by combining at least two of said audio signals, and then to control said audio output unit to generate said sound by playing said compound signal.

11. The system of claim 1, wherein said control member is configured to receive at least one of a pre-recorded audio signal defining a first content basis and a first voice basis and a real-time audio signal defining a second content basis and a second voice basis of at least one of said user and a third party, to generate at least one synthesized signal by altering at least one of said content basis and voice basis of said signal, and to control said audio output unit to generate said sound through playing said synthesized signal.

12. A pelvic relaxing system for providing tactile stimuli onto at least a portion of a pelvic structure and generating sound while controlling said stimuli through said sound, wherein said pelvic structure includes an entry as well as a wall, wherein said entry is configured to define an orifice therethrough and wherein said wall is configured to include muscles and to define an internal cavity which extends inwardly from said entry and is bound by said muscles, said system comprising:
at least one body member which is configured to have a first part for contacting said portion of said pelvic structure when engaged therewith and a second part for providing a grip to an user;
at least one actuator member which is configured to effect at least one movement of said first part for providing said stimuli onto said portion of said structure through said movement; and
at least one control member which is configured to include at least one of a sensor unit and at least one input unit, to receive an user input defining at least one dynamic pattern, and to effect said movement of said first part in response to said user input and at least partially based upon said sound.

13. The system of claim 12, wherein said control member is further configured to play said sound while effecting said movement.

14. The system of claim 12, wherein said control member is configured to store a plurality of pre-recorded audio signals from at least one of said user and a third party, to retrieve at least one of said signals, and to manipulate said actuator member and at least one dynamic feature of said movement of said first part effected thereby based upon said retrieved audio signal.

15. The system of claim 12, wherein said control member is configured to obtain an audio signal of said user and to manipulate said actuator member and at least one dynamic feature of said movement of said first part effected thereby based on said obtained audio signal.

16. The system of claim 12, wherein said control member is configured to receive a pre-recorded audio signal of at least one of said user and a third party through an external audio device and then to manipulate said actuator member and at least one dynamic feature of said movement of said first part effected thereby based upon said obtained audio signal.

17. The system of claim 12, wherein said control member is configured to receive a real-time audio signal of a third party with an external communication device and to manipulate said actuator member and at least one dynamic feature of said movement of said first part effected thereby based on said obtained audio signal.

18. The system of claim 12, wherein said control member is configured to play said sound while propagating acoustic waves of said sound toward said portion, thereby providing said stimuli to said portion not only by said movement of said first part but also by said acoustic waves.

19. A method of providing tactile stimuli effected by at least one part of a body member of a pelvic relaxing system to at least a portion of a pelvic structure while playing sound in response to operation of said system, wherein said pelvic structure includes an entry and a wall, wherein said entry forms therethrough an orifice, and wherein said wall is comprised of muscles and defines an internal cavity which extends inwardly from said entry and which is bound by said muscles, said method comprising the steps of:
engaging said body member with said pelvic structure;
supplying at least one user input to said system;
effecting at least one movement of said body part in response to said user input; and
generating said sound in response to at least one of said engaging, supplying, and effecting.

20. The method of claim 19 further comprising the step of:
synchronizing said sound with at least one of said engaging, supplying, and effecting.
